(19) United States
(12) Patent Application Publication
(10) Pub. No.: US 2015/0268248 A1
(43) Pub. Date: Sep. 24, 2015

(54) CELL SELECTIVE PROTEOME LABELING

(71) Applicant: MEMORIAL SLOAN-KETTERING CANCER CENTER, New York, NY (US)

(72) Inventors: Nicholas Gauthier, New York, NY (US); Chris Sander, New York, NY (US); Martin Miller, Cambridge (GB)

(73) Assignee: MEMORIAL SLOAN-KETTERING CANCER CENTER, New York, NY (US)

(21) Appl. No.: 14/426,596

(22) PCT Filed: Sep. 5, 2013
(86) PCT No.: PCT/US13/58212

§ 371 Date: Mar. 6, 2015

Related U.S. Application Data
(60) Provisional application No. 61/697,584, filed on Sep. 6, 2012.

(51) Int. Cl. G01N 33/68; G01N 33/50

(52) U.S. Cl. CPC G01N 33/6848; G01N 33/5005; G01N 2458/15

(57) ABSTRACT

The invention relates to a method for the Cell Type specific labeling with Amino acid Precursors (CTAP). In particular, the disclosed method permits the incorporation of stable isotope-labeled amino acids into the proteome of a vertebrate cell that has been engineered to express an exogenous enzyme that enables the cell to produce an essential amino acid from its amino acid substrate. The method employs stable isotope-labeled amino acid substrate/precursors from which essential amino acids bearing the label are generated. The labeled amino acids generated by the transgenic cell not only supports growth but specifically labels proteins of the transgenic cell. Furthermore, the use of different populations of cells expressing different exogenous amino acid-producing enzymes permits differential labeling of the proteomes of the individual cell populations in multicellular environments.

### CROSS-REFERENCE TO RELATED APPLICATIONS

This application claims the priority of U.S. provisional application No. 61/697,584 filed Sep. 6, 2012, the contents of which are incorporated herein by reference in their entirety into the present disclosure.

### SEQUENCE LISTING

The instant application contains a Sequence Listing; the file, in ASCII format, is designated 3314011AWO_SequenceListing_ST25.txt and is 5.92 kilobytes in size. The file is hereby incorporated by reference in its entirety into the instant application.

### TECHNICAL FIELD OF THE INVENTION

The present invention relates generally to cell signaling, proteome labeling, and the differential labeling of cellular proteins in mixed cell populations.

### BACKGROUND OF THE INVENTION

Cell-to-cell communication, whether mediated by direct contact or through secreted factors, is fundamental to a range of biological phenomena including tissue development, homeostasis, and pathogenesis. Recent technological advances in mass spectrometry (MS) allow for the unbiased identification of hundreds to thousands of proteins in a single sample. Labeling techniques, such as SILAC and iTRAQ, further provide relative quantitation between samples. Several limitations of current methods pose significant challenges for detecting and quantifying the molecules, such as proteins, involved in cellular communication. For example, unfortunately, such quantitative methods require samples be grown and labeled in isolation, making studies of cell-to-cell cross communication difficult. Moreover, antibody-based methods for staining proteins of interest in individual cells are low-throughput and depend on prior knowledge. On the other hand, unbiased and high-throughput approaches, such as mass spectrometry (MS), cannot distinguish proteins derived from different cell-types and therefore require samples to be grown and labeled in isolation.

Protein signal transduction induced by cell-cell interactions is difficult to investigate with current research methods. Antibodies are widely used for identification and differentiation of proteins specific to different cell types in tissue or co-culture (e.g., immunostaining or FACS sorting), however antibody-based methodologies are relatively low throughput, vary in specificity, and are biased by preselection of protein readout and availability of reagents. High-throughput and unbiased methodologies, such as MS-based proteomics, may overcome these limitations. Unfortunately, as it is unable to differentiate proteins derived from different cell types in complex cell mixtures, MS is not well suited for cell-cell communication studies. A notable example of these limitations is the inability of any method to identify the cell-of-origin of growth factors, cytokines, and other secreted proteins.

In a different approach, each distinct cell type is labeled in isolation (e.g., SILAC using L-Lysine or L-Arginine isotopes), and the fully labeled cells are subsequently mixed. Peptides identified in MS/MS can then be assigned a source cell-type from the isotopic label status. Two recent reports demonstrate the feasibility of such an approach for identifying early ephrin signaling responses and determining proteins transferred between cell types. Unfortunately, these labels become diluted as cells grow in co-culture (approximately 50% with every cell division), making this experimental setup primarily useful for investigating early proteomic events. Given the caveats of each of these methodologies, in the field of cell signaling, there is a great need in the art for novel methods that overcome the limitations with current antibody and MS-based proteomics.

What is needed is a methodology that holds the potential to address a variety of questions not easily answered with current methods, including distinguishing the cell-of-origin for secreted factors in co-culture, identifying signaling pathway alterations in multicellular environments and identifying biomarkers in vivo that are relevant to disease by linking them to the cells from which they originate.

### SUMMARY OF THE INVENTION

The present method, designated CTAP for Cell type specific labeling with Amino Acid Precursors, provides for the replacement of one or more essential amino acids required for cell growth, normally supplemented in the growth media, with stable isotopically labeled essential amino acid(s) that can be generated by the cell from stable isotopically labeled precursors of the essential amino acid. Transgenic expression by cells of interest of enzymes that catalyze substrate/precursor-to-amino acid reactions enables the selective and continuous labeling of those cells in culture or in vivo, for example, in a transgenic animal.

In one aspect, therefore, the invention relates to a method for labeling proteins in a vertebrate cell, the method comprising, exposing, under growth conditions, a transgenic vertebrate cell, i.e., one that has been engineered to express an exogenous enzyme that enables the cell to generate an essential amino acid from an amino acid precursor/substrate, to a composition comprising said amino acid precursor/substrate for a period of time sufficient for protein synthesis to occur. The substrate/precursor contains a stable isotope label, which is present in the resulting amino acid produced by the cell and ultimately, present in the proteome of the cell. Once labeled, recovery of labeled proteins from the cell(s), and evaluation of the proteins that contain the labeled amino acid facilitate investigations of the proteome of that cell and others in its environment.

In another aspect, the invention relates to a method for monitoring protein synthesis in a vertebrate cell, the method comprising a) exposing a transgenic vertebrate cell that expresses an exogenous enzyme, which enables the cell to generate an essential amino acid from the essential amino acid substrate/precursor to a composition comprising said amino acid substrate/precursor for a period of time sufficient for protein synthesis to occur, wherein said substrate/precursor for said essential amino acid comprises a stable-isotope label; b) isolating proteins from the cell; wherein proteins synthesized by said cell comprise the stable isotope-labeled essential amino acid.

In yet another aspect, the invention relates to a method for the differential labeling of cellular proteins in multiple cell types/populations, the method comprising, co-culturing a first transgenic vertebrate cell that expresses a first exogenous enzyme that can generate a first essential amino acid from a first amino acid precursor, and a second transgenic vertebrate cell that expresses a second exogenous enzyme that can generate a second essential amino acid from a second amino acid precursor, in a medium comprising a first essential amino acid precursor and a second essential amino acid, wherein said first and second essential amino acids differ only in mass, isolating proteins from said first and second vertebrate cells, and evaluating the proteins, wherein the protein can be attributed to the cell in which it was synthesized, based on its mass.

In a related aspect, the invention relates to method for differentiating proteins from a mixed population of vertebrate cells, the method comprising: (a) exposing (i) a first transgenic vertebrate cell that expresses an exogenous enzyme capable of converting a precursor/substrate for an essential amino acid to the essential amino acid; and (ii) a second vertebrate cell to a composition comprising said precursor/substrate for said essential amino acid, said precursor comprising a first stable isotope, and an essential amino acid comprising a second stable isotope for a period of time sufficient for protein synthesis to occur; (b) recovering proteins from said first and second vertebrate cells; (c) determining the amount of said first and second stable isotopes in said proteins to determine cell of origin, wherein a protein containing said first stable isotope was synthesized by said first transgenic vertebrate cell and a protein comprising said second stable isotope was synthesized by said second vertebrate cell. In some embodiments, the second vertebrate cell is also a transgenic cell that expresses an enzyme different from the enzyme expressed by the first vertebrate cell.

A method for determining the proteome of origin for proteins from a mixed cell culture, the method comprising: (a) exposing (i) a first transgenic vertebrate cell that expresses an exogenous enzyme capable of converting an essential amino acid substrate/precursor to an essential amino acid; and (ii) a second vertebrate cell; to a composition comprising said essential amino acid and said amino acid substrate/precursor for a period of time sufficient for protein synthesis to occur; wherein each of said essential amino acid and said essential amino acid substrate/precursor is labeled a different stable isotope; (b) recovering proteins from the co-cultured cells; c) determining the amount of each of said stable isotopes in the proteins; wherein proteins from said first transgenic vertebrate/mammalian cell exhibits a different mass than the proteins from said second vertebrate cells; and d) evaluating the proteins that comprise the labeled amino acid.

In yet another aspect, the invention relates to a method for the differential labeling of proteins in more than one cell population, the method comprising: (a) providing a first vertebrate cell population capable of synthesizing a first essential amino acid from a first amino acid precursor and a second vertebrate cell population capable of synthesizing a second essential amino acid from a second amino acid precursor; (b)

co-culturing said first and second vertebrate cell populations in a medium comprising said first and second essential amino acid precursors for a time sufficient for protein synthesis to occur; (c) recovering proteins from said cells; and (d) determining the amount of protein comprising said first essential amino acid in and the amount of protein comprising said second essential amino acid, wherein protein comprising the first essential amino acid was synthesized by said first cell population and protein comprising the second essential amino acid was synthesized by said second cell population. First and second precursors have different masses, for example, heavy and light lysine precursors so as to be distinguishable once incorporated into protein.

In yet another aspect, the invention relates to vertebrate cells that have been transiently or stably transfected to express an enzyme capable of producing a labeled essential amino acid from its labeled substrate/precursor, as well as novel cells and vectors containing nucleic acids encoding an exogenous enzyme for transfecting the cells.

In another aspect, the invention relates to vectors useful for the production of transgenic cells that express an exogenous enzyme that generates an essential amino acid from an essential amino acid substrate/precursor, and stable isotopically-labeled essential amino acid substrate/precursors.

In a related aspect, the invention relates to kits for labeling proteins and monitoring protein synthesis, the kit comprising vectors for the transfection of vertebrate cells so that the cells express an exogenous enzyme that generates an essential amino acid from an essential amino acid substrate/precursor and/or stable isotopically-labeled essential amino acid substrate/precursors.

In related aspects, the invention relates to methods for labeling proteins, monitoring protein synthesis and differentiating proteins in different cell types in mammalian cells. Mammalian cells are typically transiently or stably transfected to express an exogenous enzyme that produces an essential amino acid from a substrate/precursor molecule. By supplying a substrate/precursor for the essential amino acid that is labeled, not only can the transfected cell generate its own source of essential amino acid, proteins produced by these cells are labeled during synthesis.

### BRIEF DESCRIPTION OF THE DRAWINGS

FIG. 1 is a schematic showing the underlying principle of the Cell Type specific labeling with Amino acid Precursors (CTAP). (a) The CTAP methodology takes advantage of vertebrate cells' inability to produce essential amino acids, resulting in the requirement that these molecules be supplemented in culture media or diet for cell growth. In one embodiment, the method employs one of these amino acids, L-Lysine, and enzymes used to produce it from precursor molecules. By expressing exogenous L-Lysine-producing enzymes, transgenic cells can produce their own supply of L-lysine and (b) can be labeled selectively by supplementing the medium with heavy isotope-labeled forms of the lysine precursors. (c) Expressing distinct L-Lysine-producing enzymes in different cell types enables continuous cell-selective proteome labeling when grown in media lacking L-Lysine but containing the differentially-labeled precursors. (d) CTAP can be used to investigate inter- and intra-cellular signaling in a mixture of cells relevant for a range of biological phenomena involving cell-to-cell communication, including, but not limited to, development, differentiation, and pathogenesis.

FIG. 2 shows an embodiment in which vertebrate cells expressing the L-Lysine biosynthesis enzyme diaminopimelate decarboxylase (DDC) from Arabidopsis Thaliana grow on meso-2,6-diaminopimelate (DAP) in mono- and co-culture in vitro. (a) mouse fibroblast 3T3 that stably express DDC (upper panel) and (b) human breast carcinoma MDA-MB-231 cells that stably express lyr were plated in L-Lysine-free medium supplemented with 10 mM DAP, 4 mM D-Lysine, both precursors, or 0.798 mM L-Lysine. Control (empty vector) cells are shown in the lower panels. Cell growth, assessed with impedance (a correlate of the number of cells) using the xCELLigence system, was normalized to maximum growth. Error bars represent the standard deviation of three biological replicates.

FIG. 3 shows that vertebrate cell lines expressing L-Lysine biosynthesis enzymes incorporate L-Lysine produced from their precursors. At the start of the experiment, cell lysates were collected from (a) DDC-expressing 3T3 cells labeled heavy (H) and (b) lyr-expressing MDA-MB-231 cells labeled light (L) (top panels). Cells were further passaged and samples harvested after 13 days in L-Lysine-free media containing the indicated precursors (bottom panels). Label status of Lysine-containing peptides was assessed by quantitative LC-MS/MS and percent incorporation of heavy label was determined using H/L ratios from MaxQuant analysis (median peptide=dashed bar with percentages indicated).

FIG. 4 shows that there are limited molecular changes in precursor versus L-Lysine conditions. (a) DDC-expressing 3T3 cells were plated in SILAC media supplemented with DAP, L-Lysine or neither (starved). After 72 hours, mRNA was harvested and profiled for gene expression levels using the Illumina microarray platform. Expression differences of DAP versus L-Lysine (left panel) and starved vs L-Lysine (right panel) are plotted as a function of statistical significance (moderated t-statistics adjusted for multiple testing by the Benjamini and Hochberg method). Highlighted genes (green) are more than 2-fold differentially regulated at the level of FDR<0.05. (b) As in (a) except MDA-MB-231 cells expressing lyr were plated on L-Lysine, D-Lysine or in starved conditions. All experiments were performed in triplicate.

FIG. 5 shows that using two distinct enzyme-precursor pairs, co-cultured cells exhibit precursor-based differential proteome labeling. (a) DDC-expressing 3T3 cells (mouse) were labeled with heavy L-Lysine (H) and lyr-expressing MDA-MB-231 cells (human) with light L-Lysine (L) and mixed prior to sample analysis by LC-MS/MS (upper panel). The same cells were co-cultured and analyzed after 3 passages on DAP (L) and D-Lysine (H) (lower panel). Peptides unique to the mouse or human proteome are green and red, respectively. (b) GFP⁺ HEK293T expressing DDC were co-cultured with mCherry⁺ MDA-MB-231 cells expressing lyr in media containing DAP (L) and D-Lysine (H). Separate LC-MS/MS runs of sorted GFP⁺ (upper panel) and mCherry⁺ (lower panel) cells were performed and identified proteins are shown. Median indicated by blue line. (c) Proteins derived from unsorted co-culture of cells as in (b). Highlighted are proteins unique to each transgenic cell line (GFP and DDC in HEK293T, mCherry and lyr in MDA-MB-231 cells). Mean of transgenes for each HEK293T (DDC/GFP) and MDA-MB-231 (lyr/mCherry) are indicated with green and red lines, respectively.

FIG. 6 demonstrates an application of CTAP for determining cell-of-origin for secreted factors. (a) DDC-expressing 3T3 cells (mouse) and lyr-expressing MDA-MB-231 cells (human) were co-cultured in DAP (L) and D-Lysine (H). Prior to sample collection, cells were grown for 24 hours in serum-free media and the supernatant (media) was collected. After concentrating proteins by ultracentrifugation and methanol-chloroform extraction, the sample was analyzed by LC-MS/MS. Only proteins containing identified peptides which are unique to mouse (green) and human (red) are displayed. (b). Similar to (a) except co-culture consisted of two human cell lines: HEK293T expressing DDC and MDA-MB-231 cells expressing lyr. Colors depict relative protein abundance as determined by SILAC quantitation of mixed mono-culture. Uncolored points represent proteins that were not identified in the mono-culture sample. Annotated are the five proteins with highest H/L ratios: Galectin-3BP (LGALS3BP, Q08380); Serpin A3 (SERPINA3, P01011); Cartilage-link protein (CRTL1, P10915); Osteonectin (SPARC, P09486); Cathepsin X (CTSZ, Q9UBR2). Underlined proteins were identified in a recent study of MDA-MB-231 secreted proteins (18).

FIG. 7 is a diagram showing examples of L-lysine producing enzymes and their substrates. Several enzymes have been found in bacteria, fungi, and plants that catalyze reaction leading to the production of L-Lysine from precursor compounds. Four examples of these enzymes and their respective precursors are indicated.

FIG. 8 are graphs showing growth of human HEK 293T and mouse 3T3 cell lines on L-Lysine and different precursors of L-Lysine. (a) Cells were seeded in 96 well format using at least 4 replicates per condition and cell proliferation was measured with the Resazurin (AlamarBlue) assay at the time indicated. Note that both cell lines stop growing when no L-Lysine is present, confirming that mammalian cells are L-Lysine auxotrophic. Cells show no or limited growth response when the medium is supplemented with high (mM-range) concentrations of the L-Lys precursors meso-2,6,-diaminopimelate (DAP, b), N-□-cbz-L-Lys (Z-Lys, c), and D-Lysine (D-Lys, d). In contrast, both cell lines exhibit substantial growth response when the medium is supplemented with N2-acetyl-L-Lys (N2A, e).

FIG. 9 are graphs showing that HEK293T cells expressing the L-Lysine biosynthesis enzyme diaminopimelate decarboxylase (DDC) specifically grow on meso-2,6-diaminopimelate (DAP). HEK293T cells stably transfected with DDC (left panel) or empty control vector (right panel) were cultured in 0.798 mM L-Lysine, 10 mM DAP, or neither (blank). Cell growth was estimated by the impedance-based xCelligence assay and data was normalized to the maximum value for each cell-type. Note that only HEK293T cells that express DDC grow on DAP. Error bars represent the standard deviation of three biological replicates.

FIG. 10 are graphs showing that 3T3 cells expressing the CBZcleaver enzyme grow on Z-Lysine and partially incorporate L-Lysine produced from Z-Lysine (CBZ-Lysine). (a) 3T3 cells stably trasfected with CBZcleaver (left panel) or empty control vector (right panel) were cultured in 0.798 mM L-Lysine, 2.5 mM Z-Lysine, or without either (blank). Cell growth was estimated by the impedance-based xCELLigence assay and data was normalized to maximum values for each cell-type. Error bars represent the standard deviation of three biological replicates. (b) Peptide histograms depicting the heavy (K8), medium (K4), and light (K0) status of the 200 most intense peptides (that contain L-Lysine) in CBZcleaver-expressing 3T3 cells. The labeling status was assessed by quantitative LC-MS/MS at the beginning of the experiment where the cells were labeled with medium L-Lysine (left, K4) and after 10 days in L-Lysine-free media with heavy labeled Z-Lysine (right, Z8). The percent label incorporation for the median peptide is indicated (red bars). Concentration of L-Lysine (K4) used was 0.798 μM, and Z-Lysine (Z8) was 2.5 mM. Note that, although specific to CBZcleaver-expressing cells, both growth on Z-Lysine and L-Lysine incorporation based on Z-Lysine were incomplete, and therefore, we discontinued further experimentation with the CBZcleaver-Z-Lysine enzyme-precursor pair.

FIG. 11 are graphs showing that limited mRNA expression differences were observed on growth of precursor versus L-Lysine (a) 3T3 cells expressing DDC were plated on L-Lysine, DAP, or in DAP/L-Lysine free (starved) conditions. After 72 hours, mRNA was harvested and run on the Illumina microarray platform. Representative arrays of three biological replicates are shown. Black dots represent genes that change more than two-fold between conditions. Dashed lines depict boundaries for 2-fold expression differences between samples. (b) Similar to (a) except MDA-MB-231 cells expressing lyr were plated on L-Lysine, D-Lysine, or in starved conditions.

FIG. 12 are graphs showing that cells grown on precursors exhibit few or no protein abundance changes relative to those grown on L-Lysine. (a) DDC-expressing 3T3 cells were grown on either 10 mM DAP, 0.798 mM L-Lysine-4 (K4), or 0.798 mM L-Lysine-8 (K8), and analyzed by LC-MS/MS. Using label-free quantitation by the MaxQuant software, the intensities of the top 200 most intense proteins (minimum two peptides quantified) were compared between the conditions. Pearson correlation coefficients and r-squared values are provided. Intensity ratios greater than 2 are indicated (black dots). (b) Similar to (a) except lyr-expressing MDA-MB-231 were grown on 4 mM D-Lysine, 0.798 mM L-Lysine, or 0.798 mM L-Lysine-4 (K4). Note that the correlation between cells grown on precursor versus L-Lysine (left panels) is similar to that of cells grown on two different stable isotopes of L-Lysine (SILAC-labeled biological replicate, right panels).

FIG. 13 are graphs showing that drug perturbation induces comparable effects to cell viability for both cells on DAP versus L-Lysine and enzyme-expressing versus empty-vector control cells. In the upper panel, DDC-expressing 3T3 cells were grown in the presence of either 10 mM DAP (green) or 0.798 mM L-Lysine (blue) in various concentrations of drugs as indicated (target of drug is indicated in parenthesis). Cell viability was measured after 48 hours of drug exposure with AlamarBlue and normalized to untreated control cells. The lower panel compares DDC-expressing 3T3 cells (green) to empty vector control cells (blue) in the presence of 0.798 mM L-Lysine.

FIG. 14 are Western blots showing that molecular response to starvation, FBS stimulation, and drug perturbation are largely similar for both cells on DAP versus L-Lysine as well as enzyme-expressing versus empty-vector control cells. In the upper panel, DDC-expressing 3T3 cells were grown in the presence of either 10 mM DAP or 0.798 mM L-Lysine in media with 10% FBS (basal), without FBS (serum-starved), starved for 24 h and stimulated with 10% FBS for 1 h (FBS), or stimulated with FBS and perturbed with 5 μM AKT Inhibitor VIII (EMD Chemicals) for 1 h (FBS+AKTi). In the lower panel, DDC-expressing 3T3 cells and empty vector control cells were grown in the presence of 0.798 mM L-Lysine and exposed to similar conditions. For both experiments, cells were lysed and the response of several phosphoproteins was assessed by western blotting. Loading is indicated with GAPDH. Two biological replicates are shown.

FIG. 15 are graphs showing that using two distinct enzyme-precursor pairs, co-cultured cells grow on precursors in L-Lysine free conditions and maintain similar proportion over several passages. DDC-expressing 3T3 GFP⁺ cells were plated with lyr-expressing MDA-MB-231 mCherry⁺ cells and the media was supplemented with 10 mM DAP and 4 mM D-Lysine in L-Lysine-free conditions. The co-cultures were split 3 times (1:15) and the ratio of GFP⁺ and mCherry+ was determined at each passage using image-based flow cytometer (Tali, Invitrogen). A representative fluorescent microscopic image at passage 3 is depicted.

FIG. 16 are graphs showing that lowering the concentration of D-Lysine decreases background labeling in co-cultures. DDC-expressing 3T3 cells were plated with lyr-expressing MDA-MB-231 cells and the media was supplemented with 10 mM DAP-0 (L) and 2.5 mM D-Lysine-8 (H). A lysate sample was collected after 3 passages (13 days in culture) and analyzed by LC-MS/MS for labeling status of L-Lysine containing peptides (left). Using the same sample, peptide intensity is plotted against the H/L ratio (right). Only peptides that are unique to the mouse (green) or human (red) proteome by sequence are analyzed. Note that lowering the concentration of D-Lysine to 2.5 mM from previous used levels (4 mM, FIG. 4) decreases the amount of unspecific labeling in DDC-expressing 3T3 cells, possibly due to reduced L-Lysine sharing between cells in the co-culture or other factors.

FIG. 17 show post sort FACS analysis of co-cultured human HEK293T and MDA-MB-231 cells. GFP⁺ HEK293T expressing DDC were co-cultured with mCherry⁺ MDA-MB-231 cells expressing lyr and sorted for GFP⁺ and mCherry⁺ cells by FACS. In a post-sort analysis, purity of each of the sorted populations were assessed by flow cytometry for the same fluorophores. Percentages are indicated. Note that, although a post-sort analysis of the sorted populations showed a high enrichment for the expected fluorophores, there were approximately 2-5% cross-contamination.

FIG. 18 shows that label status of differentially labeled co-culture cells shows good agreement with SILAC-labeled mono-cultures. (a) HEK293T expressing DDC cells were co-cultured with MDA-MB-231 cells expressing lyr in 10 mM DAP (L) and 4 mM D-Lysine (H). Cell lysate was collected, proteins were digested and subjected to LC-MS/MS. Colors depict relative protein abundance as determined by quantitation (median-centered H/L ratios) of mixed mono-cultures of similar cells that were separately labeled using standards SILAC labeling. Uncolored points represent proteins that were not identified in the mono-culture sample. (b) Co-culture H/L ratios were binned and the average mono-culture H/L ratio in each bin was determined and depicted using a similar color scheme as in (a). (c) Correlation between mono-culture and co-culture H/L ratios.

FIG. 19 are graphs showing that label status of secreted proteins of differentially labeled co-culture cells shows good agreement with SILAC-labeled mono-cultures. (a) HEK293T expressing DDC cells were co-cultured with MDA-MB-231 cells expressing lyr in 10 mM DAP (L) and 4 mM D-Lysine (H). 24 hours prior to harvest of supernatant, cells were grown in serum-free medium and proteins were concentrated by ultra-centrifugation and methanol-chloroform extraction. Proteins were digested and subjected to LC-MS/MS. The quantified H/L ratios of the secreted proteins are compared to median-centered H/L ratios from mixed mono-cultures of similar cells that were separately labeled using standard SILAC labeling. Co-culture H/L ratios were binned and the average mono-culture H/L ratio in each bin was determined. Note that a relatively high proportion of the proteins identified with high H/L ratios could not be identified intracellularly. (b) Correlation between mono-culture and co-culture H/L ratios.

FIG. 20 are graphs showing cell-selective labeling of co-cultures using one enzyme-precursor pair. (a) Co-culture of DDC expressing GFP⁺3 T3 cells and empty vector control mCherry⁺3 T3 cells with (left panel) or without (right panel) 10 mM DAP and various concentrations of L-Lysine. After 72 h in co-culture, flow cytometry was used to determine the number of GFP⁺ and mCherry⁺ cells. Date points represent at least two biological replicates. (b) Mouse 3T3 cells expressing DDC were labeled with K8 and co-cultured in 40 μM K8 and 10 mM DAP along with K4 labeled human MDA-MB-231 cells. The first co-culture lysate sample was taken immediately after mixing of the cells (seeding) and the second sample was taken after two passages. The labeling status of peptides unique to the mouse or human proteome are displayed separately; ambiguous peptides were ignored.

FIG. 21 shows an embodiment in which the growth of HEK293T cells expressing a truncated Lysine racemase (lyr) from P. mirabilis on D-lysine in mono- and co-culture in vitro is comparable to growth on L-lysine. Cell growth, assessed with impedance (a correlate of the number of cells) using the xCELLigence system, was normalized to maximum growth. Error bars represent the standard deviation of three biological replicates.

FIG. 22 shows an embodiment in which growth of MDA-MB-231 cells expressing truncated Lysine racemase (lyr) from P. mirabilis on D-lysine in mono- and co-culture in vitro is comparable to growth on L-lysine.

FIG. 23 shows an embodiment in which the growth of B16 cells expressing truncated Lysine racemase (lyr) from P. mirabilis on D-lysine in mono- and co-culture in vitro is comparable to growth on L-lysine.

### DETAILED DESCRIPTION

All publications, patents and other references cited herein are incorporated by reference in their entirety into the present disclosure.

In practicing the present invention, many conventional techniques in molecular biology are used. Such techniques are well known and are explained in, for example, Sambrook et al., 2001, Molecular Cloning: A Laboratory Manual, Third Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins, eds.); Transcription and Translation, 1984 (Hames and Higgins, eds.); Animal Cell Culture, 1986 (R. I. Freshney ed.); Immobilized Cells and Enzymes, 1986, (IRL Press); Perbas, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively); Current Protocols in Molecular Biology, John Wiley & Sons, Inc. (1994), and all more current editions of these publications. The contents of these references and other references containing standard protocols, widely known to and relied upon by those of skill in the art, including manufacturers' instructions are hereby incorporated by reference as part of the present disclosure.

In the description that follows, certain conventions will be followed as regards the usage of terminology.

The term “expression” refers to the transcription and translation of a structural gene (coding sequence) so that a protein (i.e. expression product) having biological activity is synthesized. It is understood that post-translational modifications may remove portions of the polypeptide that are not essential and that glycosylation and other post-translational modifications may also occur.

The term “transfection,” as used herein, refers to the uptake, integration and expression of exogenous DNA by a host cell, and includes, without limitation, transfection with plasmids, episomes, other circular DNA forms and other vectors and transfectable forms of DNA known to those of skill in the art. The expression vector may be introduced into host cells via any one of a number of techniques known in the art including but not limited to viral infection, transformation, transfection, lipofection or other cationic lipid based transfection, calcium phosphate co-precipitation, gene gun transfection, and electroporation. These techniques are well known to persons of skill in the art.

Atoms with the same atomic number (proton number) but different mass numbers (the sum of the proton and neutron numbers) are called isotopes. Isotopes result from the presence in an atom of additional neutrons and include radioactive and stable isotopes. A “stable isotope,” therefore, refers to an isotope of an element that is not radioactive. Examples of stable isotopes include, for example, stable isotopes of carbon (e.g. ¹³C), hydrogen (e.g. ¹H and ²H, deuterium), oxygen (e.g., ¹⁷O and ¹⁸O), nitrogen (¹⁵N) and sulfur (³³S, ³⁴S, ³⁸S.) Stable isotopes are used in the method of the invention to impart a detectable difference in mass to the protein/proteome in which the isotope becomes incorporated.

A “stable isotope-” or “stable isotopically-labeled” amino acid or amino acid precursor, therefore, is an analog of the amino acid/precursor which incorporates a stable isotope. Examples of labeled substrate/precursors include without limitation, light (unlabeled) meso-2,6-diaminopimelate (DAP0, Sigma), light (unlabeled) D-Lysine (Sigma), medium [²H₄]D-Lysine (DLYS4, C/D/N Isotopes), heavy [²H₈]D-Lysine (DLYS8, C/D/N Isotopes), heavy labeled [¹³C₆, ¹⁵N₂] Z-Lysine etc.

“Relative abundance” as that term is known in mass spectrometry is a method of reporting the amount of each Mass to Charge measurement (m/z) after assigning the most abundant ion 100%. All of the other peaks are reported as a relative intensity to the largest peak.

The present method represents a technological advance in that it allows researchers to distinguish cell-types (and their proteomes) in a mixture of cells by engineering certain of the cells for continuous and specific metabolic labeling by introducing a nucleic acid encoding an amino acid-producing enzyme, thereby allowing the cell to overcome its normal auxotrophic state. Stable isotope labeling by amino acid precursors in vivo or in cell culture is a simple and straightforward approach for incorporation of a label into proteins of the transgenic cells for mass spectrometry (MS)-based quantitative proteomics.

Basically, the method relies on metabolic incorporation of a given ‘light’ or ‘heavy’ form of an amino acid into the proteins. The method relies on the incorporation into the cell's proteins of amino acids with substituted stable isotopic nuclei (e.g. deuterium, ¹³C, ¹⁵N etc.) that are produced by the cell from a stable isotopically-labeled amino acid precursor.

One or more cell populations that exist in the same environmental niche or which are co-cultured in vitro are exposed to different amino acid precursors that contain a different makeup of stable isotopes (e.g., light vs. heavy precursors, the end-product of which becomes ¹²C and ¹³C labeled L-lysine) so that the amino acids generated from them are distinguishable by mass spectrometry because they have different masses. When the labeled analog of an amino acid precursor is supplied to cells that have the ability to produce the amino acid from a stable isotopically-labeled precursor, the corresponding stable isotopically-labeled amino acid is incorporated into all newly synthesized proteins. After a number of cell divisions, each instance of this particular amino acid will be replaced by its isotope labeled analog.

In the presence of stable isotopically-labeled substrate/precursor for the essential amino acid, selective labeling of these cells in conjunction with modern tandem (LC-MS/MS) facilitates the differentiation, identification, and quantification of proteins derived from each cell type in a mixed population of cells.

In one aspect, the invention relates to a method for labeling proteins in a vertebrate cell, the method comprising, exposing, under conditions permitting growth/protein synthesis, a vertebrate cell that has been engineered to be able to generate an essential amino acid from its amino acid precursor/substrate, to a composition comprising said amino acid precursor/substrate for a period of time sufficient for protein synthesis to occur. The substrate/precursor contains a stable isotope label, which is present in the resulting amino acid and ultimately in proteins synthesized in the presence of the labeled amino acid. Once labeled, recovery of the proteins from the cell, and evaluation of the proteins that comprise the labeled amino acid are possible. In one embodiment, the essential amino acid is lysine and substrate/precursors therefore include diaminopimelate (DAP), D-lysine and Z-lysine. Lysine substrate/presursors contain at least one stable isotope of carbon, hydrogen, oxygen, and/or nitrogen.

In one aspect, therefore, the invention relates to a method for labeling proteins in a vertebrate cell, the method comprising, exposing, under growth conditions, a vertebrate cell that has been engineered to be able to generate an essential amino acid from its amino acid precursor/substrate, to a composition comprising said amino acid precursor/substrate for a period of time sufficient for protein synthesis to occur. The substrate/precursor contains a stable isotope label, which is present in the resulting amino acid produced by the cell and ultimately the proteome of the cell. Once labeled, recovery of labeled proteins from the cell(s), and evaluation of the proteins that contain the labeled amino acid facilitate investigations of the proteome of that cell and others in its environment.

In one embodiment, the essential amino acid is lysine and substrate/precursors therefore include without limitation diaminopimelate (DAP), D-lysine and Z-lysine. Lysine substrate/precursors contain at least one stable isotope (or no stable isotopes in the case of light label) of carbon, hydrogen, oxygen, and/or nitrogen. Any combination of stable isotopes may be present in a particular form of the essential amino acid, as long as each amino acid has a different mass and is therefore distinguishable, for example, by mass spectrometry analysis, from other forms of the same essential amino acid. Examples of labeled substrate/precursors include without limitation, light meso-2,6-diaminopimelate (DAP0, Sigma), heavy [²H₈]D-Lysine (DLYS8, C/D/N Isotopes), heavy labeled [¹³C₆, ¹⁵N₂] Z-Lysine etc.

In certain embodiments, a vertebrate cell is transiently or stably transfected to express one or more enzyme components of the synthetic pathway for the essential amino acid. Enzymes may be encoded by nucleic acids from an exogenous source including bacteria, fungi, plants etc. Exemplary enzymes include without limitation, diaminopimelate carboxylase (DDC) from, for example, Arabidopsis thaliana or Escherichia coli, lysine racemase (lyr) from, for example, Proteus mirabilis and CBZcleaver, for example, from Sphingomonas paucimobilis.

The following represent examples of applications of the disclosed technology. Numerous other applications of the disclosed method are feasible.

In this work, the validity and feasibility of the CTAP method for cell-selective proteome labeling in multicellular systems is demonstrated. Using precursors of the essential amino acid L-Lysine and enzymes that catalyze its synthesis, this disclosure shows that canonical amino acids can be isotopically labeled in specific cell types in co-culture. Cell types of both mouse and human origin successfully overcome L-Lysine auxotrophy in the presence of specific enzyme-precursor pairs involved in the production of L-Lysine. The work demonstrates that there are limited molecular and phenotypic consequences of culturing DDC or lyr-expressing cells in L-Lysine free conditions on DAP or D-Lysine, respectively. Mass spectrometry analysis of enzyme-expressing cells in monoculture shows complete molecular labeling by L-Lysine derived from precursor. Differential-labeling of individual cell types in co-culture can be achieved using a dual-enzyme-precursor pair setup in the absence of L-Lysine, allowing all identified proteins to be assigned relative-quantitated values in each cell type. Supporting these data, it was also found that CTAP is applicable for labeling a specific cell-type of interest in a mixed cell culture system using only one enzyme-precursor pair, although titrating down the amount on L-Lysine in the media is necessary (for example). Finally, analyzing the supernatant of cells in co-culture, cell-of-origin of secreted proteins can be readily established.

There are several features of the CTAP system that collectively distinguish it from other cell-selective protein labeling approaches. First, the products of enzymatic catalysis are canonical amino acids allowing mature proteins to maintain their normal structure and avoiding possible functional alterations when using amino acid analogs. Second, CTAP allows individual cell populations to be continuously labeled as they are grown and passaged over extended periods of time. Third, the genetic requirement of enzyme activity to overcome essential amino acid auxotrophy makes labeling controllable by limiting transgenic expression. Fourth, utilizing multiple enzyme-precursor pairs permits differential labeling of multiple distinct cell types simultaneously. Fifth, CTAP can distinguish proteins from different cell types of the same organism rather than relying on artificial inter-species experimental setups. Finally, CTAP makes use of the same previously developed data-analysis workflows as the widely used SILAC method. To the best of our knowledge, CTAP is the only method in which the proteome of specific cell populations can be labeled continuously and differentially by canonical amino acids in a complex mixture of cells.

CTAP can be quickly adaptable across many cell types without phenotypic or molecular disturbance. Cell lines that are suitable for use in practicing the method of the invention include, but are not limited to mouse fibroblast 3T3 cells, mouse melanoma, B16 cells, human embryonic kidney (HEK) cells, human mammary adenocarcinoma cells, MDA-MB-321, etc.

Focusing on the DDC/DAP and lyr/D-Lysine enzyme-precursor pairs, the results indicate that cells behave similarly when cultured on their specific precursor relative to L-Lysine, however, these similarities were measured after a period of growth that varied in length depending on the cell type tested.

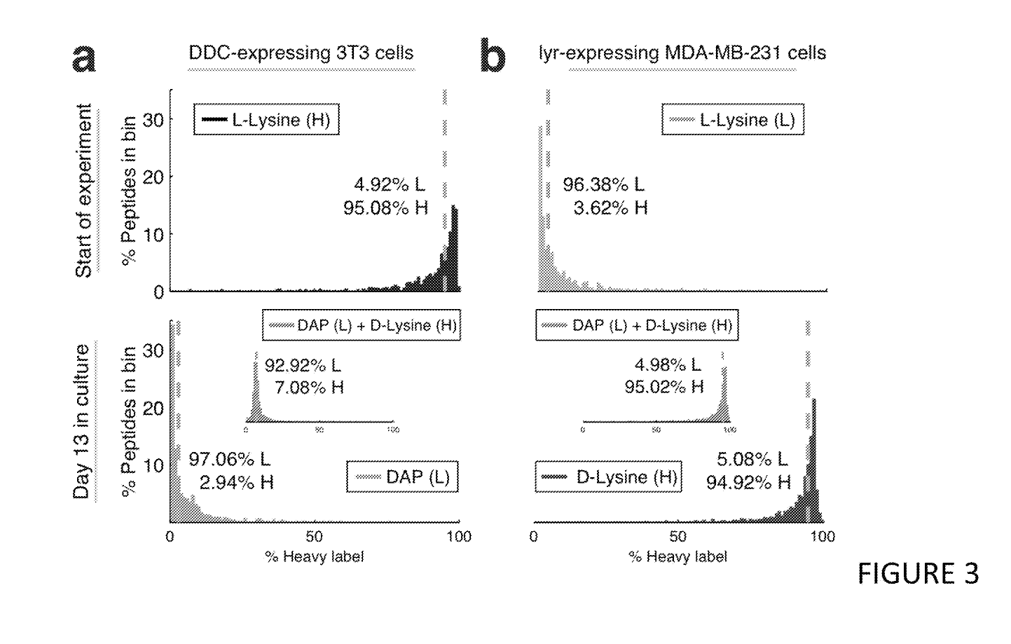

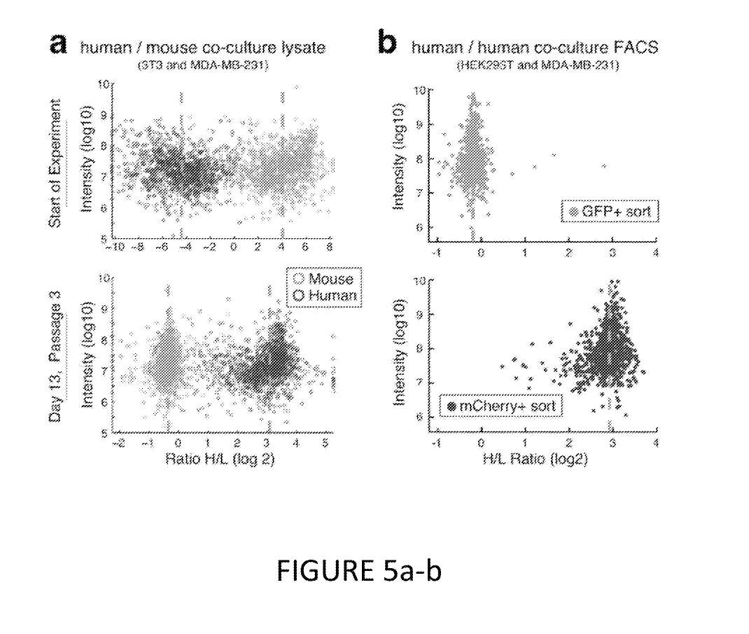

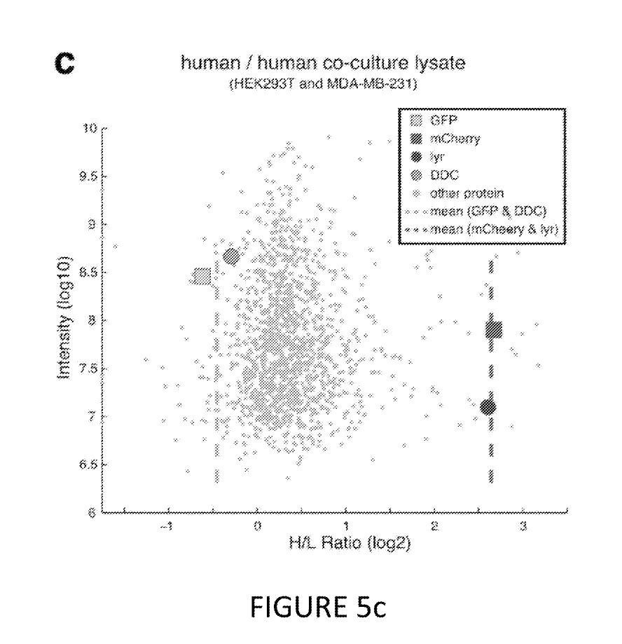

The principle of proteome labeling by amino acids produced from stable isotope-labeled precursors was demonstrated in mono-culture. Although this labeling was complete for both precursor-enzyme pairs (approximately 95%, FIG. 3), when cells were combined into co-culture we observed suboptimal labeling in one of the populations (approximately 50%, FIG. 5). There are several possible explanations for this discrepancy. First, cells in co-culture might share amino acids or transfer proteins that are further metabolized. Second, phagocytosis might lead to amino-acid transfer. Third, transgenic enzymes may have extracellular activity. A combination of these possibilities or other unknown mechanisms may lead to the observed background labeling and will be addressed in future studies. Although desired, in this case complete labeling was not necessary to determine relative protein expression levels between each cell type.

It is anticipated that CTAP will be an important tool for gaining insight into intercellular signaling in fundamental processes of but not limited to organogenesis, maintenance, and disease development. For example, in various cancers the interaction between malignant cells and the surrounding stromal tissue has been shown to be important for disease progression, maintenance, and altered drug efficacy (19-21). How stromal cells affect these processes is unclear, partly due to inadequate techniques for assaying their roles. The use of CTAP may address these limitations and offer an opportunity to understand the molecular mechanisms by which surrounding stroma alter tumor growth and response to treatment. Once precursor delivery, tolerance, and enzyme expression are optimized, another possible application of CTAP will be identification of disease biomarkers in vivo. Current approaches for biomarker identification are limited by their inability to classify whether a potential marker originates from the diseased tissue itself or from normal tissue. Using the described technique we can circumvent these limitations, as proteins from specific cell types of interest can in principle be labeled continuously in vivo. Any labeled protein identified in the serum or proximal fluids will originate from the cell type of interest.

Utilization of exogenous amino acid biosynthesis components allows for continuous cell-selective metabolic labeling of proteins. Furthermore, the principle behind CTAP can be applied to essential amino acids other than L-Lysine. CTAP therefore, represents a significant step forward in the field of proteomics, allowing unbiased and high-throughput MS/MS to differentiate peptides derived from distinct cells in complex cellular mixtures. The method is a powerful tool which will allow researchers to probe a variety of questions regarding cell-cell communication and cell-specific origin of biomarkers not easily accessible with other methodologies.

Investigating protein signal transduction induced by secreted factors and cell-cell interactions is limited by current research methods. A notable example of these limitations is the inability of any current method to identify the cell-of-origin of growth factors, cytokines, and other secreted proteins. Antibodies are widely used for identification and differentiation of proteins specific to different cell types in tissue or co-culture (e.g., immunostaining or fluorescence-activated cell sorting, FACS), however antibody-based methods are relatively low throughput, vary in specificity, and are biased by preselection of protein readout and availability of reagents. High-throughput and unbiased methods, such as quantitative mass spectrometry (MS) based proteomics (1-3), might overcome some of these limitations. However, as MS is unable to differentiate from which cell-type proteins originate in complex cell mixtures, it is not well suited for cell-cell communication studies. Research in cell-cell communication would greatly benefit from methods that overcome the complimentary limitations with current antibody and MS-based proteomics.

Several recent efforts have been made to differentiate the proteome of distinct cell types in co-culture. In one such approach each distinct cell type is labeled in isolation (e.g., using heavy stable isotope-labeled L-Lysine or L-Arginine), and the fully labeled cells are subsequently mixed. Peptides identified in liquid chromatography tandem mass spectrometry (LC-MS/MS) can then be assigned a source cell-type from the isotopic label status. Two recent reports demonstrate the feasibility of such an approach for identifying early ephrin signaling responses (4) and determining proteins transferred between cell types (5). Unfortunately, these labels become rapidly diluted as cells grow and divide in co-culture, making this experimental setup primarily useful for investigating early proteomic events. In a different approach, protein sequence differences between species are used to determine cell-of-origin in cross-species co-cultures and xenografts (6; 7). Although this approach has the ability to distinguish proteins between cell types, the major drawbacks are that only a subset of peptides can be differentiated, established same-species co-culture models cannot be used, and the findings from mixed-species models may not be physiologically relevant. Yet another technique utilizes tRNA-synthetases that specifically recognize and incorporate amino acid analogs into proteins (8; 9). Using certain tRNA-synthetase/amino-acid-analog pairs, this method provides for both proteomic incorporation that is specific to transgenic cells as well as the ability to perform affinity enrichment on chemical moieties (e.g., azides). However, structural differences between the analogs and canonical amino acids might cause unpredictable functional alterations in mature proteins (10). Given the caveats of each of these methods, a novel method for continuous cell-specific labeling with canonical amino acids would be valuable.

The present invention provides a method for cell-selective proteomic labeling that overcomes the problems of throughput and specificity of antibody-based cell staining, possible functional perturbations induced by amino acid analogs, physiological relevance of cross-species models, and the requirement of short co-culture time frames for cells labeled in isolation. This technique allows the proteome of distinct cell-types growing together either in vivo or in co-culture to be differentially labeled by canonical amino acids, which leads to naturally folded proteins and avoids the use of amino acid analogs. Our method utilizes the inability of vertebrate cells to synthesize certain amino acids required for growth and homeostasis. These “essential” amino acids are produced in some plants, bacteria, and lower eukaryotes, and must be supplemented to the media of vertebrate cultured cells or obtained in the diet of animals (11). Using transgenic expression of enzymes that synthesize essential amino acids, vertebrate cells are able to overcome auxotrophy by producing their own amino acids from supplemented precursors. These precursors can be isotopically-labeled, allowing cell-of-origin of proteins to be determined by label status identified by LC-MS/MS. For these studies we focus on L-Lysine, as the biosynthesis of this essential amino acid is well studied and it is commonly used in quantitative proteomic methods such as stable isotope labeling by amino acids in cell culture (SILAC) (2). In this work, we test the validity and feasibility of the CTAP method and demonstrate its viability for continuous and differential metabolic labeling of cells in co-culture. Using this novel method, we are able to determine relative protein expression between two cell types in co-culture and identify cell-of-origin of secreted proteins.

### Examples

The invention, having been generally described, may be more readily understood by reference to the following examples, which are included merely for purposes of illustration of certain aspects and embodiments of the present invention, and are not intended to limit the invention in any way.

### Engineering Vertebrate Cells to Grow on L-Lysine Precursors

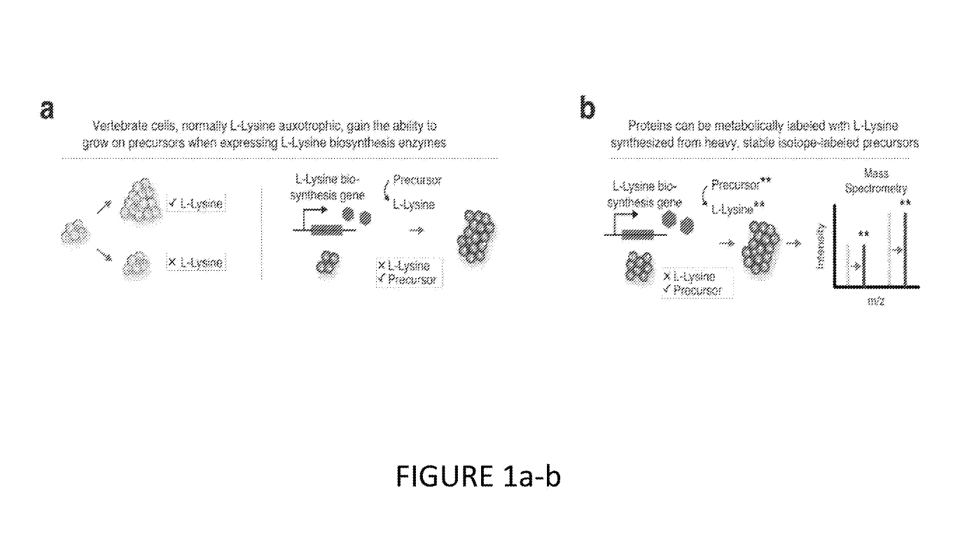

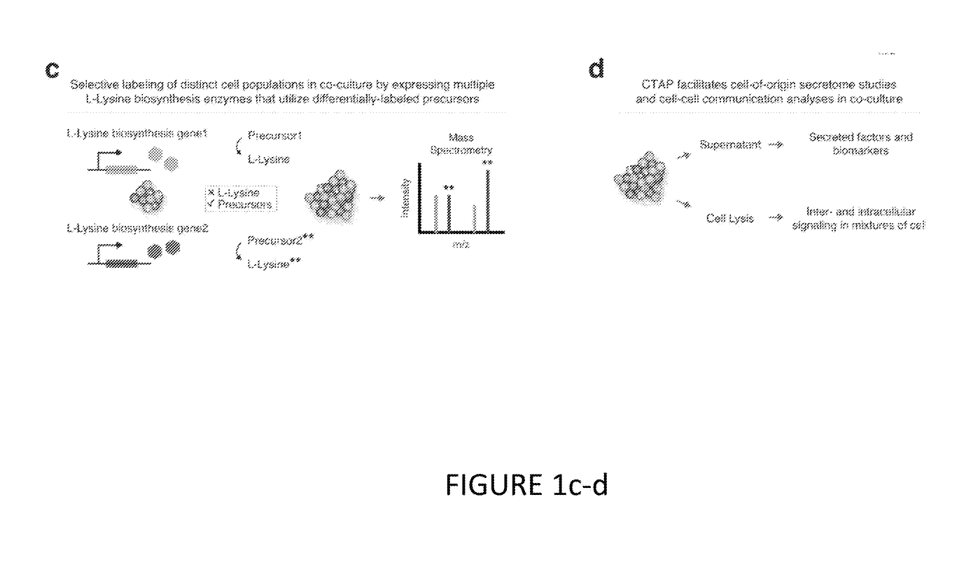

By engineering vertebrate cells to produce their own supply of L-Lysine from labeled precursors, it is possible to achieve differential proteomic tagging of specific cell types in co-culture (FIG. 1a-d). The first step was to identify a set of substrate/precursor-enzyme pairs in which the precursor/substrate was readily available and the enzyme had no described orthologs in vertebrate genomes (FIG. S1). One such substrate/precursor-enzyme pair has successfully been used to rescue L-Lysine auxotrophy when creating a positive selection system for vector incorporation (12; 13). In that system, however, only cell growth was assessed.

To investigate the candidate precursors and eliminate those that autonomously rescue L-Lysine auxotrophy, growth rates in SILAC media supplemented with L-Lysine, various precursors, or in L-Lysine-free conditions were examined. With the exception of N₂-acetyl-L-Lysine, the tested precursors alone had little or no effect on growth in wild-type cells (FIG. S2a-e).

Next, whether transgenic expression of L-Lysine biosynthesis enzymes would allow cells to acquire the ability to grow on precursors was investigated. Genes encoding the enzymes diaminopimelate decarboxylase (DDC) from Arabidopsis thaliana and Lysine racemase (lyr) from Proteus mirabilis were stably expressed in several cell lines (Table 1).

TABLE 1

| cell type | | enzyme | | mass |
|---|---|---|---|---|
| (origin) | species | (origin) | precursor | difference |
| MDA-MB-231 | human | Lyr | D-Lysine | 0 Da |
| (breast) | | (P. mirabilis) | | 8 Da |
| HEK293T | human | DDC | DAP | 0 Da |
| (embryonic) | | (A. thaliana) | | |
| 3T3 | mouse | DDC | DAP | 0 Da |
| (breast) | | (A. thaliana) | | |

Abbreviations used in Table 1 are as follows: lyr=Lysine Racemase, DDC=diaminopimelate decarboxylase, DAP=meso-2,6-diaminopimelate. Da=Dalton. * indicates the heavy form of the precursor (deuterated, 3,3,4,4,5,5,6,6-d8).

Additionally, 3T3 and HEK293 cells were produced that express CBZcleaver and truncated lyr, respectively. Other transgenic cells generated that successfully overcome lysine auxotrophy include B16 expressing either DDC or truncated lyr, and MDA-MB-231 cells that express DDC or truncated lyr.

### Generation of DDC Constructs

The gene for diaminopimelate decarboxylase (DDC) from Arabidopsis thaliana was cloned directly from A. thaliana cDNA using the primers in Table 2. The oligonucleotide sequence for DDC is given in SEQ ID NO: 10. For more information about DDC in A. thaliana, see AT3G14390 at the Arabidopsis Information Resource (TAIR).

Three PCR reactions were performed to generate pLM-GFP-P2A-DDC for insert into pLM using the Agel and Sall restriction enzymes. In the first reaction, a GFP-P2A oligonucleotide fusion that began with an Agel site was created. The second reaction generated a PCR fragment of P2A-DDC flanked by Sall. Finally, an overlapping PCR reaction created Agel-GFP-P2A-DDC-Sall. This sequence was then ligated into the Agel-Sall digested pLM vector.

TABLE 2

| | | | |
|---|---|---|---|
| | | | Product |
| Reaction | Primer Name | Oligonucleotide Sequence (5′-3′) | Size |

Clone DDC(TAIR Id = AT3G14390) from Arabidopsis thaliana cDNA

| | | | |
|---|---|---|---|
| 1 | FWD-DDC- | GCCctcgagATGGCGGCAGCTACTCAAT | 1465 nt |
| | XhoI | (SEQ ID NO. 1) | |
| | REV-DDC- | CGCgaattcGTTCATAGACCTTCAAAGAAACGC | |
| | EcoRI | | |
| | | (SEQ ID NO. 2) | |

Subclone DDC into mSCV-IRES-GFP (pMIG)

| | | | |
|---|---|---|---|
| 1 | FWD-DDC- | ATCgaattcATGGCGGCAGCTACTCAAT | 1475 nt |
| | EcoRI | (SEQ ID NO. 3) | |
| | REV-DDC- | CGCgaattcGTTCATAGACCTTCAAAGAAACGC | |
| | EcoRI | | |
| | | (SEQ ID NO. 2) | |

Subclone DDC into pLM-GFP

| | | | |
|---|---|---|---|
| 1 | FWD- | CCGGTTACCGGTATGGTGAGCAAGGGCGAGGAG | 795 nt |
| | Fluorescent | (SEQ ID NO. 4) | |
| | Gene-AgeI | | |
| | REV-P2A- | | |
| | Fluorescent | AGGGCCGGGATTCTCCTCCACGTCACCTGCTTG | |
| | Gene | TTTGAGTAGTGAGAAGTTTGTTGCTCCAGATCC | |
| | | CTTGTACAGCTCGTCCATGCCG | |
| | | (SEQ ID NO. 5) | |
| 2 | FWD-P2A-DDC | GGATCTGGAGCAACAAACTTCTCACTACTCAAA | 1533 |
| | | CAAGCAGGTGACGTGGAGGAGAATCCOGGCCCT | |
| | | ATGGCGGCAGCTACTCAAT | |
| | | (SEQ ID NO. 6) | |
| | REV-DDC-SalI | CCGGTTGTCGACTCATAGACCTTCAAAGAAACG | |
| | | CA | |
| | | (SEQ ID NO. 7) | |
| 3 | FWD- | CCGGTTACCGGTATGGTGAGCAAGGGCGAGGAG | 2262 nt |
| | Fluorescent | (SEQ ID NO. 4) | |
| | Gene-AgeI | | |
| | REV-DDC-SalI | CCGGTTGTCGACTCATAGACCTTCAAAGAAACG | |
| | | CA | |
| | | (SEQ ID NO. 7) | |

### Truncation of Lysine Racemase (Lyr)

Results from initial attempts to produce a cell expressing lysine racemase (lyr) from P. mirabilis suggested that the enzyme was being secreted by the transfected cell. In view of a determination by SignalP (data not shown) that lyr contained a signal peptide, constructs for truncated forms of the enzyme, including T18 (N-terminal 18 amino acids removed) and T12 (N-terminal 12 amino acids removed) were designed.

The oligonucleotide sequence for truncated lyr from Proteus mirabilis, as synthesized for use in some embodiments of the present method, is given in SEQ ID NO: 11. The amino acid sequence of T18 with a His-tag is given in SEQ ID NO: 14.

Three PCR reactions were performed to generate pLM-GFP-P2A-lyr for insert into pLM using the Agel and Sall restriction enzymes. Primers used are shown in Table 3. In the first reaction, a mCherry-P2A oligonucleotide fusion that began with an Agel site was created. The second reaction generated a PCR fragment of P2A-lyr flanked by Sall. Finally, an overlapping PCR reaction created Agel-mCherry-P2A-lyr-Sall. This sequence was then ligated into the Agel-Sall digested pLM vector.

TABLE 3
Subclone lyr into pLM-mCherry

| | | | |
|---|---|---|---|
| 1 | FWD- | CCGGTTACCGGTATGGTGAGCAA | 786 nt |
| | FluorescentGene- | GGGCGAGGAGAGGGCCGGGATTC | |
| | AgeI | TCCT | |
| | | (SEQ ID NO. 4) | |
| | REV-P2A- | AGGGCCGGGATTCTCCTCCACGT | |
| | FluorescentGene | CACCTGCTTGTTTGAGTAGTGAG | |
| | | AAGTTTGTTGCTCCAGATCCCTT | |
| | | GTACAGCTCGTCCATGCCG | |
| | | (SEQ ID NO. 5) | |
| 2 | FWD-P2A-lyr | GGATCTGGAGCAACAAACTTCTC | 1300 nt |
| | | ACTACTCAAACAAGCAGGTGACG | |
| | | TGGAGGAGAATCCCGGCCCTATG | |
| | | GAGCCTGGGCATCAGATAC | |
| | | (SEQ ID NO. 8) | |
| | REV-lyr-SalI | TGTTGTCGACTCAATCCACCAGC | |
| | | ACGCG | |
| | | (SEQ ID NO. 9) | |
| 3 | FWD- | CCGGTTACCGGTATGGTGAGCAA | 2020 nt |
| | FluorescentGene- | GGGCGAGGAG | |
| | AgeI | (SEQ ID NO. 4) | |
| | REV-lyr-SalI | TGTTGTCGACTCAATCCACCAGC | |
| | | ACGCG | |
| | | (SEQ ID NO. 9) | |

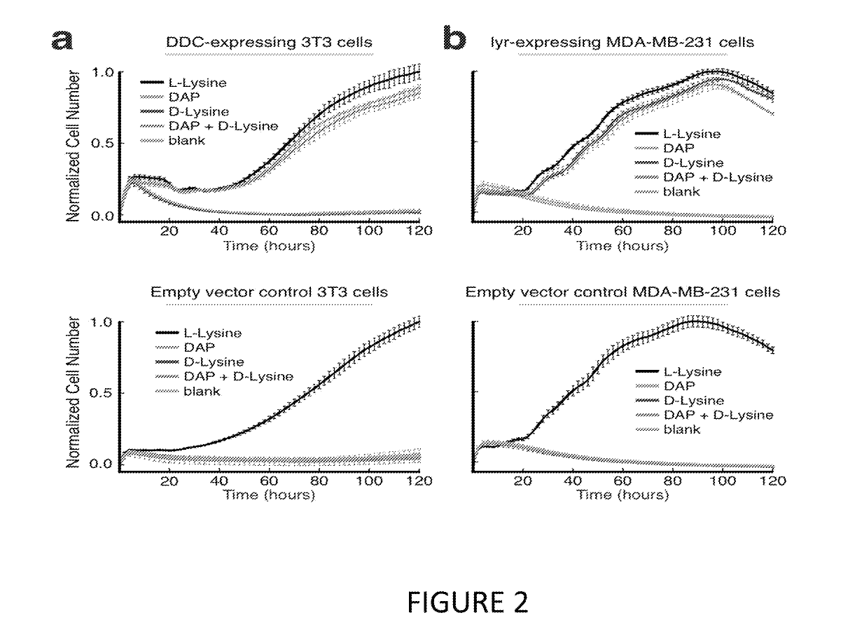

DDC-expressing mouse 3T3 and HEK293T cells, along with lyr-expressing human MDA-MB-231 cells, exhibited growth rates in media supplemented with the precursors meso-2,6-diaminopimelate (DAP) and D-Lysine, respectively, comparable to those in media containing L-Lysine (FIGS. 2a, 2b, and Figure S3). Furthermore, the enzyme-precursor pairs were specific, as no growth was observed in the cross enzyme-precursor setup or in empty-vector controls (FIGS. 2a and 2b). These mono-culture growth rescue results show that transgene-based enzymatic turnover of precursors is responsible for the growth rescue observed in L-Lysine free conditions. The time to reach normal growth rates varied between cell-types from immediate to a short passaging/selection period. In addition to DDC and lyr, we also tested and found specific growth rescue with the enzyme CBZcleaver and substrate Z-Lysine, supporting the adaptability of the CTAP method (Figure S4).

Cell-Selective Incorporation of L-Lysine Produced from Precursors

Although the phenotypic data served as a proxy for L-Lysine availability, they did not directly show molecular precursor-based incorporation. To investigate whether L-Lysine is directly produced by enzymatic-turnover of the supplemented precursors, we applied the SILAC principle of exchanging the isotopic label status of amino acids from one form to another (e.g., light L-Lysine to heavy L-Lysine) (2). At the beginning of the experiments, DDC-expressing 3T3 cells were labeled with heavy [¹³C₆, ¹⁵N₂]L-Lysine (H) and lyr-expressing MDA-MB-231 cells were labeled with light L-Lysine (L). These cells were then grown in monoculture for 13 days (3 passages) in L-Lysine-free that contained unlabeled DAP (L), heavy-labeled [²H₈]D-Lysine (H), or both precursors. Protein from cell lysate was trypsin-digested, submitted to high resolution LC-MS/MS, and H/L ratio for each peptide was determined by MaxQuant (14).

In the presence of light-labeled DAP alone, peptides identified in DDC-expressing 3T3 cells switched from being predominantly labeled heavy (95%, median peptide) to light (97%) (FIG. 3a). Similarly, the peptides identified in lyr-expressing MDA-MB-231 cells changed from 96% light to 95% heavy in the presence of heavy-labeled D-Lysine (FIG. 3b). This amount of labeling can be considered complete as it is similar to the initial H/L label status and levels typically reported in SILAC experiments (15; 16). To test the amount of unspecific labeling (i.e., cross contamination), cultures were also grown in the presence of both precursors. Supplementing the DDC precursor DAP had no effect on the label switch in lyr-expressing MDA-MB-231 cells, while the presence of D-Lysine (H) marginally increased the heavy label status (from 3% to 7%) in DDC-expressing 3T3 cells (insets, FIG. 3). This difference was possibly due to heavy L-Lysine contamination in heavy D-Lysine (95% enantiomeric purity, C/D/N Isotopes) and might be reduced with higher purity. Taken together, these data indicate that lyr and DDC-expressing cells are able to specifically incorporate and grow on L-Lysine synthesized directly from their respective precursors.

### Limited Perturbation to Cells Growing on Precursors

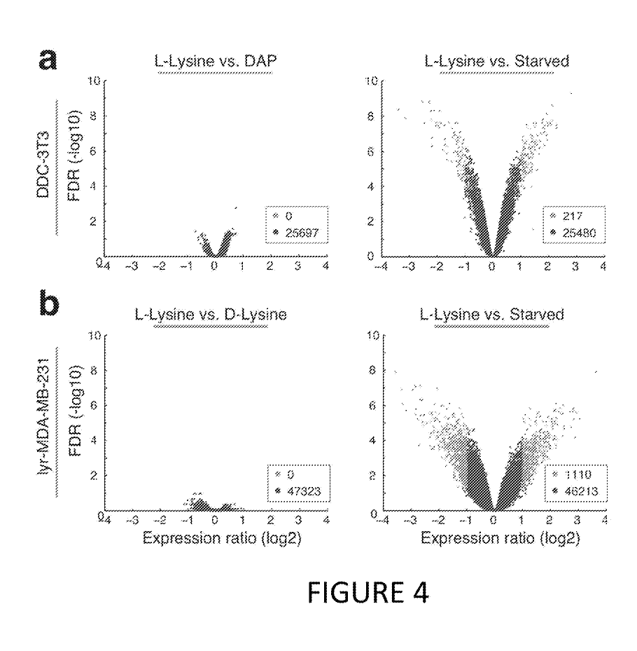

Next, whether cells behave similarly when grown on precursors compared to L-Lysine was investigated. Cells were cultured for 3 days in media containing L-Lysine, precursor, or neither (starved, positive control for perturbed state) and mRNA expression levels were profiled using microarrays (FIG. 4, Figure S5). Relative to the basal L-Lysine condition, no genes changed significantly when DDC-expressing 3T3 cells were grown on DAP, while 217 genes changed in the starved conditions (FDR<0.05 and expression ratio greater than two, FIG. 4a). The same pattern was also seen in lyr-expressing MDA-MB-231 cells when grown on L-Lysine or D-Lysine relative to starved cells (FIG. 4b). Furthermore, several assays were performed to probe the effects of precursor-based growth, including measurement of protein changes by LC-MS/MS as well as growth response to drug perturbations (Figures S6-S8). Although minor differences exist, overall these data demonstrate that growing cells on their precursors has little effect on gene expression, protein expression, or behavior.

### Continuous and Differential Proteome Labeling in Co-Culture

After demonstrating the principle of precursor-based L-Lysine production and incorporation in mono-culture, the next step was to test whether the same cells could be differentially labeled in co-culture with each population utilizing a distinct enzyme-precursor pair. To assess the specificity of labeling, we took advantage of species-specific sequence differences to compare label status between the enzyme-expressing mouse 3T3 and human MDA-MB-231 cell lines. Labeling each cell type in isolation, the 3T3 cells were initially cultured in heavy L-Lysine (H) and the MDA-MB-231 cells in light L-Lysine (L). A sample was harvested and combined 1:1 to verify the ability to differentiate label status based on species-specific peptide classification. As expected, labels of mouse-specific and human-specific peptides at the start of the experiment were confirmed to be primarily heavy and light, respectively (FIG. 5a, top panel).

With the expectation that each cell type would exchange label status, the pre-labeled cells were then combined in co-culture into media containing both DAP (L) and D-Lysine (H). After three passages, with near equal growth rates of each cell population (Figure S9), the two cell types switched labels (FIG. 5a, bottom panel). While the human MDA-MB-231 cells became predominantly labeled from heavy precursor (90% or 3.1 log 2 H/L), the mouse 3T3 cells became approximately 57% (−0.4 log 2 H/L) labeled from light precursor. For the 3T3 cells, the level of labeling was lower than expected from the results observed in mono-culture (see Figure S10). Even with this lower labeling efficiency, the mouse and human peptides exhibit a similar number of overlapping H/L ratios as the SILAC labeled monocultures (top panel contains 3.2% peptides with H/L ratios not separable by cell type versus 4.7% in bottom panel, FIG. 5a). These distinct H/L ratios in species-specific sequences therefore demonstrate the ability to differentially label the proteome across cell types in co-culture.

Having validated continuous and differential labeling of human-mouse cells in co-culture, the next step was to determine whether the CTAP method could differentiate the proteome of a same-species co-culture system. DDC-expressing GFP⁺ HEK293T cells were plated together with lyr-expressing mCherry⁺ MDA-MB-231 cells. After five days of growth in DAP (L) and D-Lysine (H), a co-culture sample was sorted for mCherry and GFP⁺ cells by FACS (Supplementary FIG. S11) and each of the sorted populations was separately subjected to LC-MS/MS. Analysis of protein from the GFP+ and mCherry⁺ cells showed similar labeling efficiency to that seen in the human-mouse co-culture, with each cell population exhibiting distinct H/L ratios (FIG. 5b). Another set of samples was collected directly from non-sorted co-cultures, subjected to LC-MS/MS, and 1362 proteins were identified. Focusing on the transgenic proteins exclusive to each cell population (GFP and DDC for the HEK293T as well as mCherry and lyr in MDA-MB-231 cells), we observed the expected H/L ratios corresponding to those determined by FACS (FIG. 5c). When analyzing all identified proteins, the H/L ratios exhibited a near-normal distribution with the transgenes lying in the tails. Although these tails contain relatively few members, they likely represent cell type specific proteins (FIGS. 5c and S12). At the depth of the proteome investigated, this result is consistent with a recent report that found most proteins are ubiquitously expressed but at different relative abundance levels (17). In summary, these results demonstrate the ability to tag the proteome in a cell-specific manner and show that label status (H/L ratio) is directly related to the relative protein abundance level between the two cell types.

### Determining Cell-of-Origin of Secreted Proteins in Co-Culture

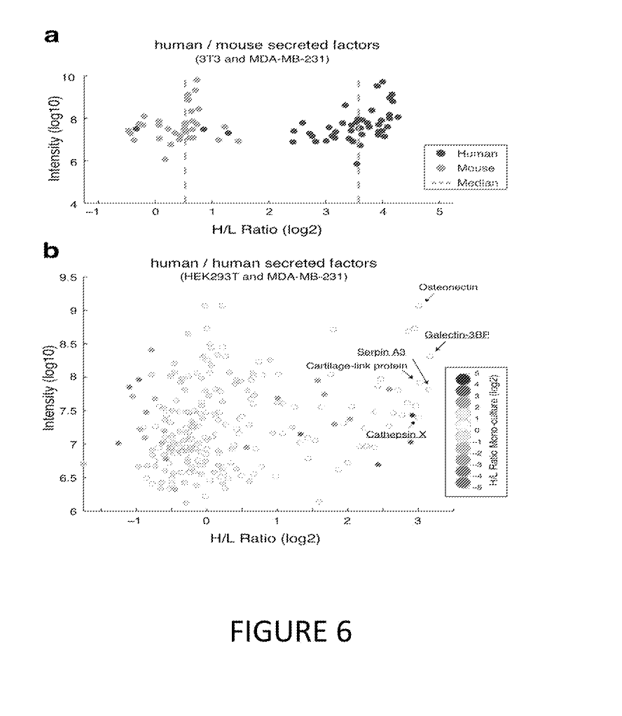

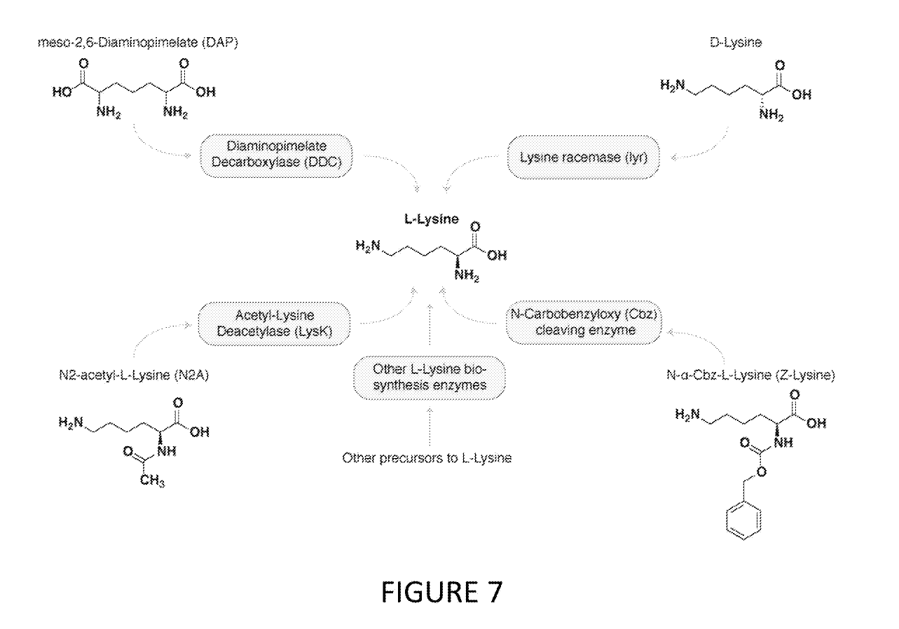

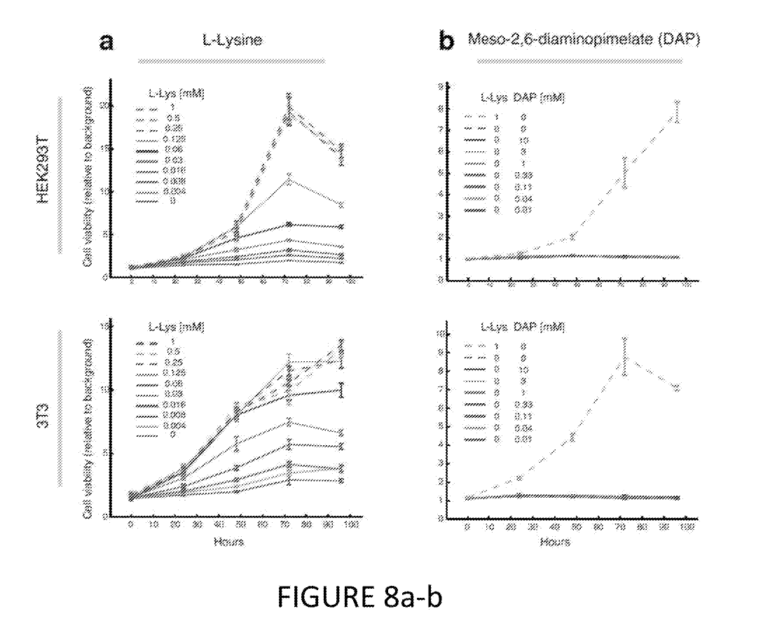

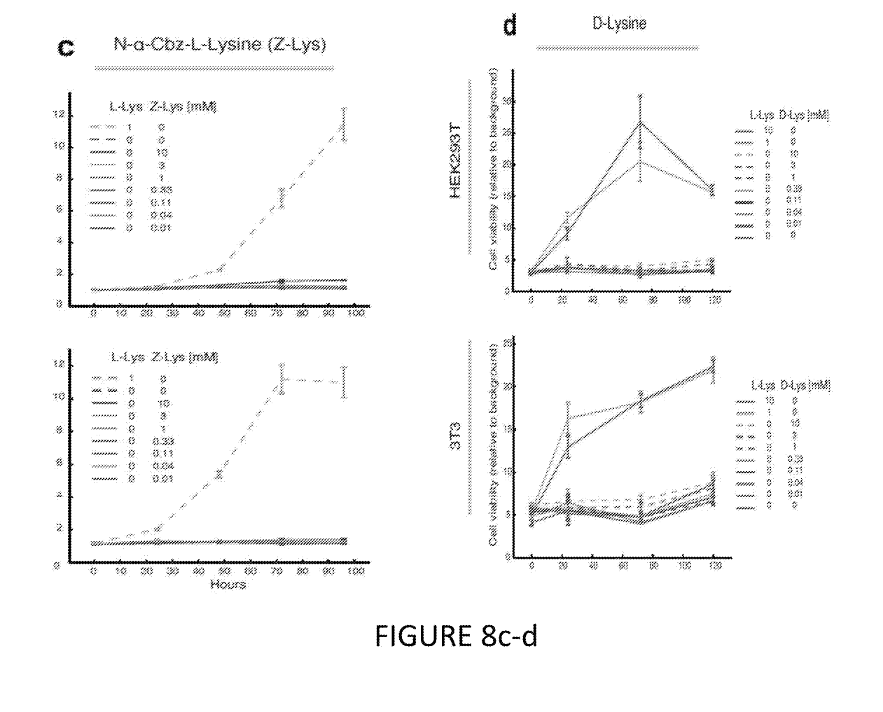

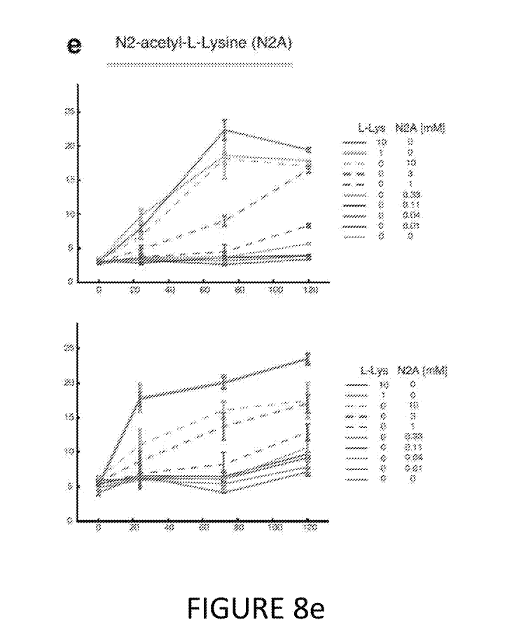

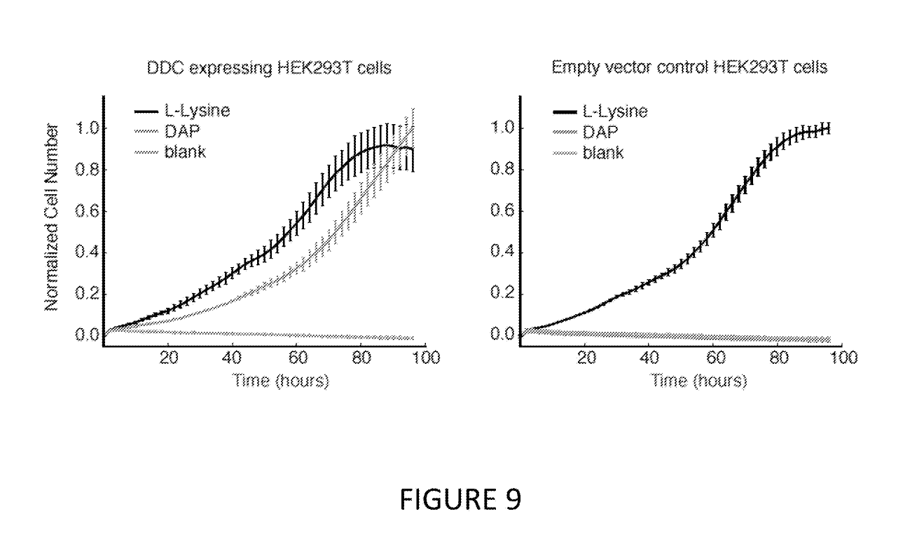

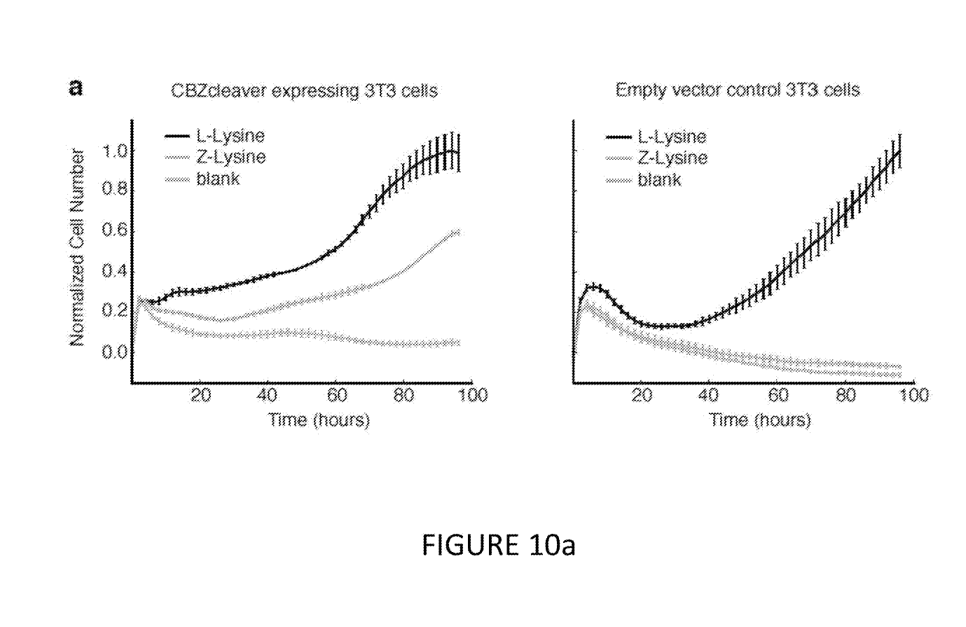

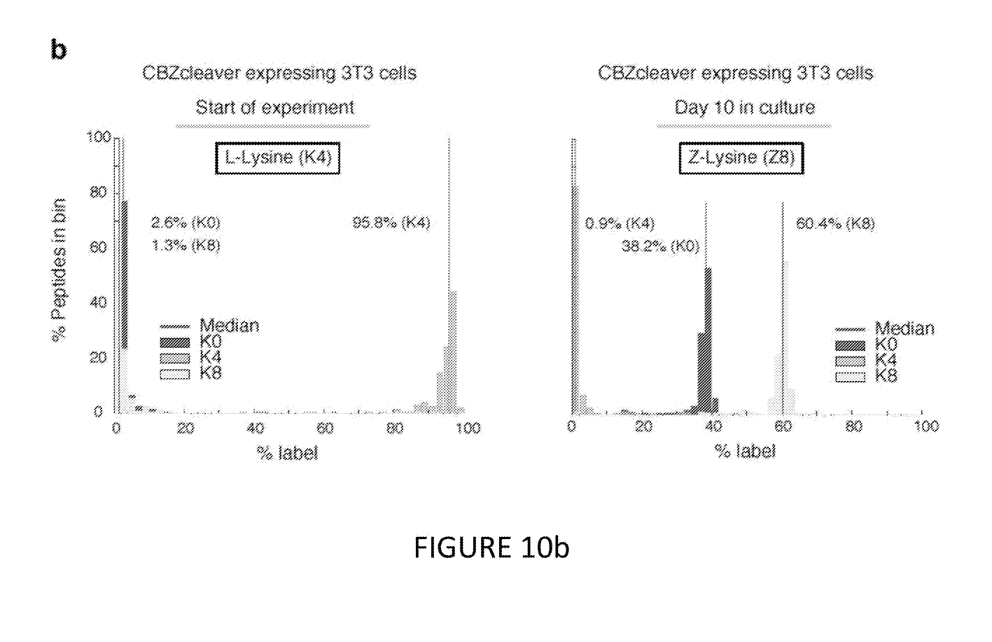

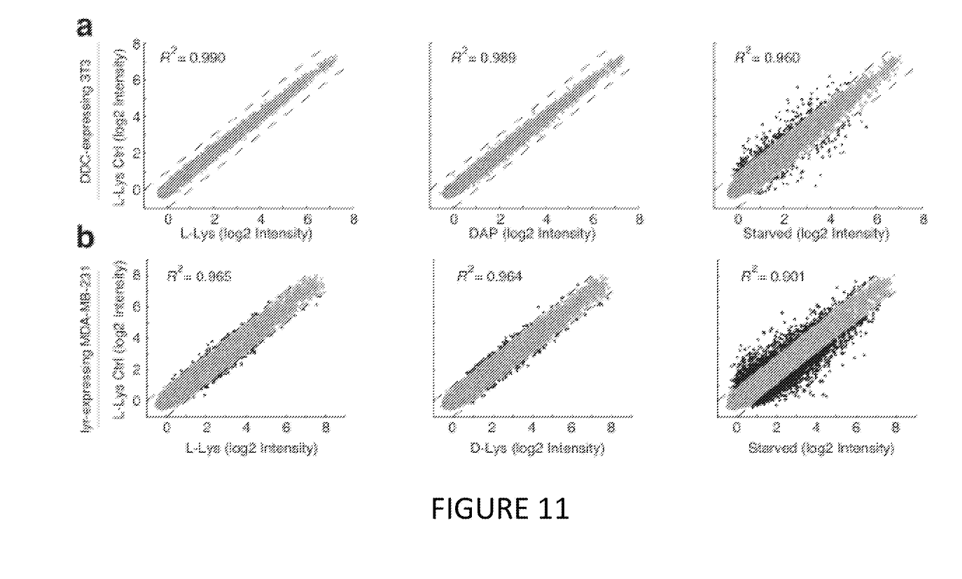

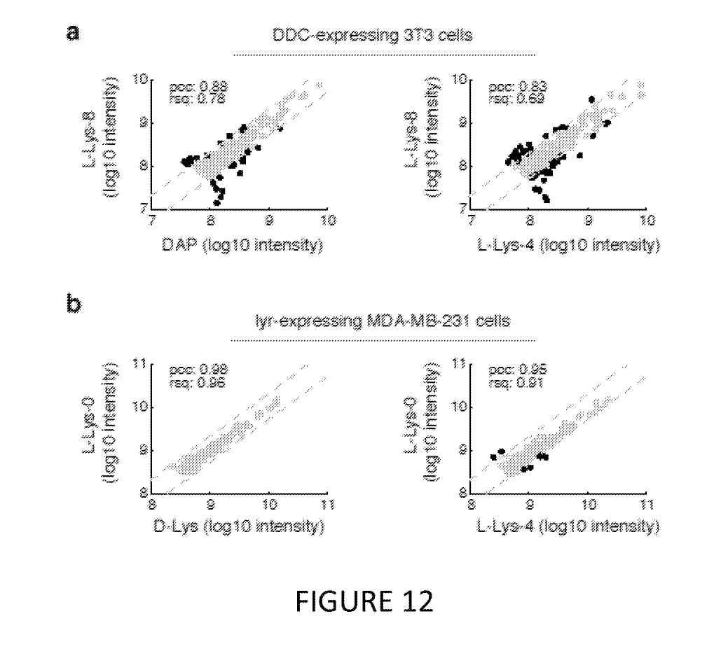

To test the unique potential of the CTAP method to discriminate the cell-of-origin of secreted factors, supernatant was collected from the same human-mouse co-culture setup as the previous section. Prior to harvesting, the cells were grown for 24 hours in serum-free media to avoid overloading the sample with serum proteins. Secreted proteins were concentrated by ultracentrifugation, precipitated by methanol-chloroform, and subjected to LC-MS/MS. Focusing on proteins identified only by species-specific peptides, nearly all species-specific proteins could be completely distinguished by label alone (FIG. 6a). These results demonstrate the ability of the method to determine cell-of-origin for secreted proteins in co-culture.

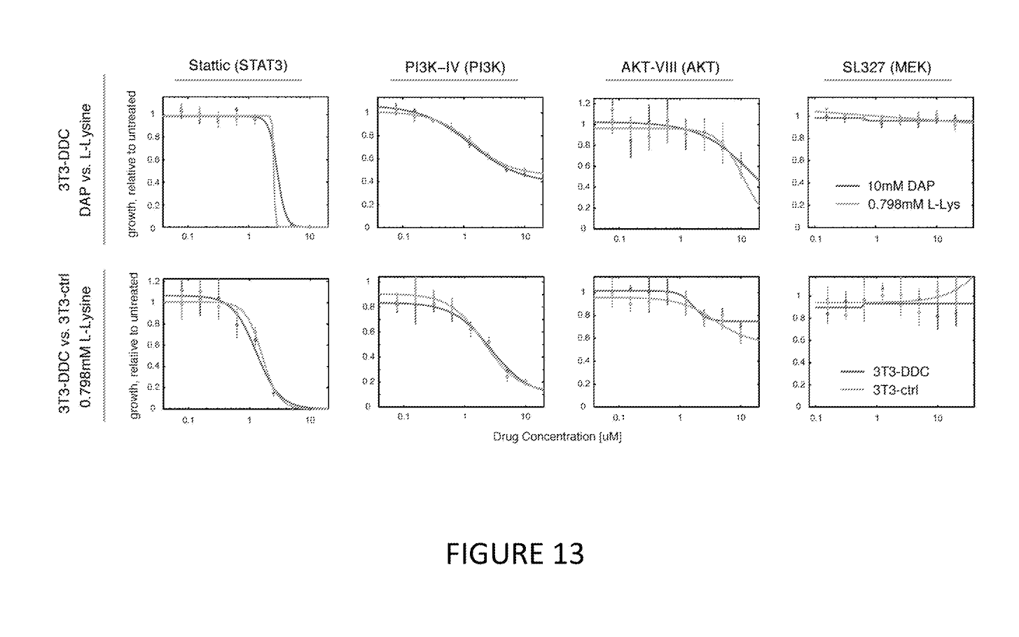

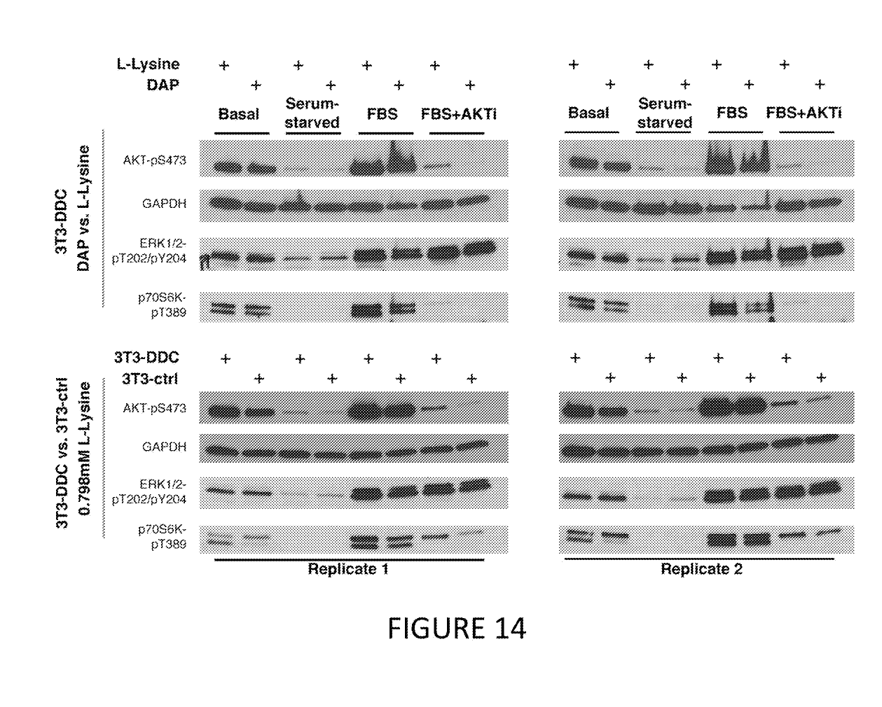

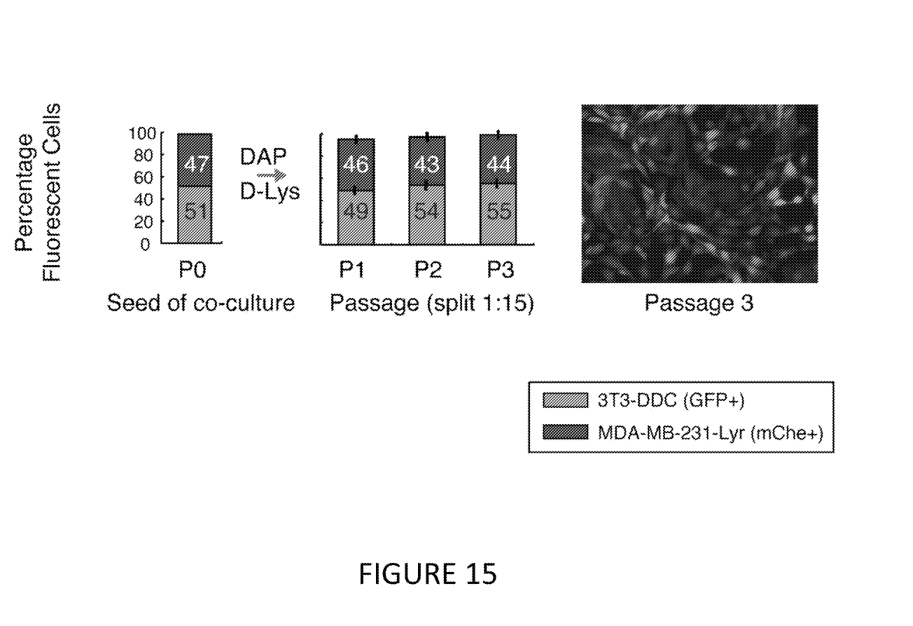

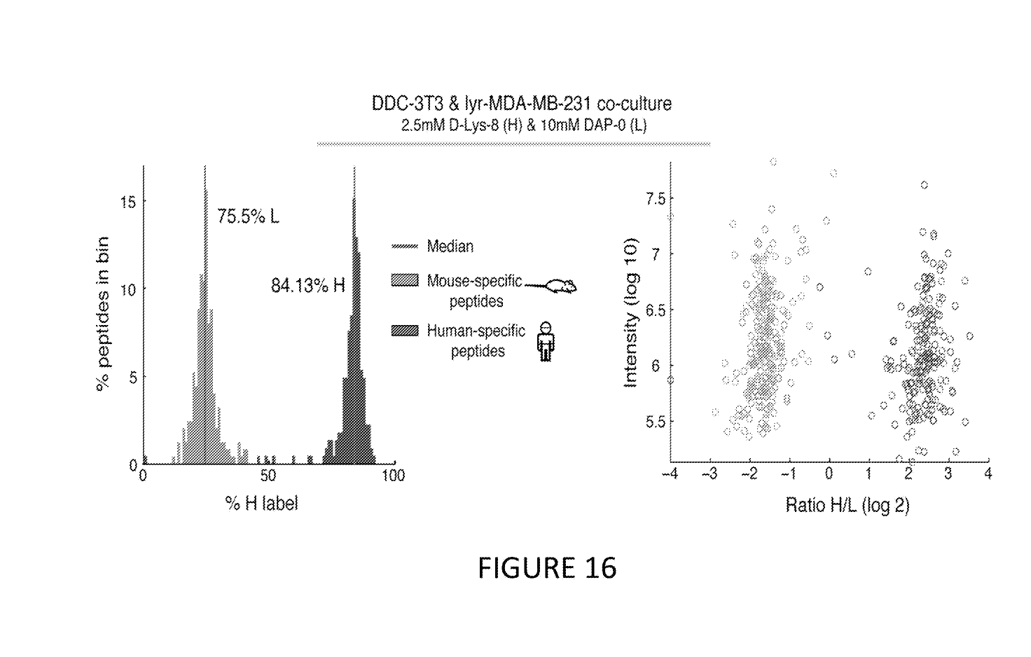

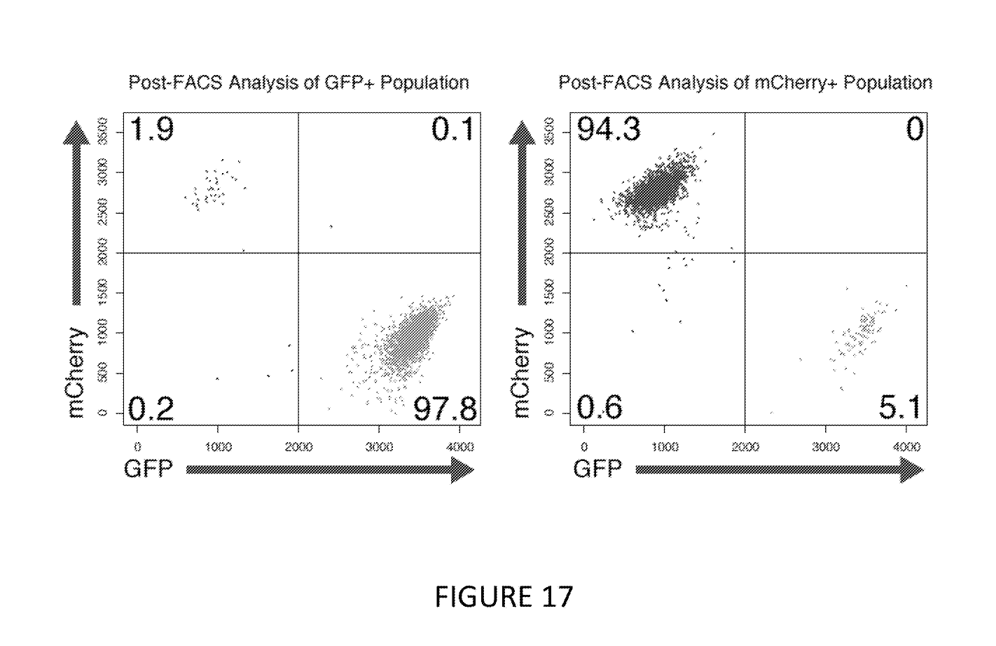

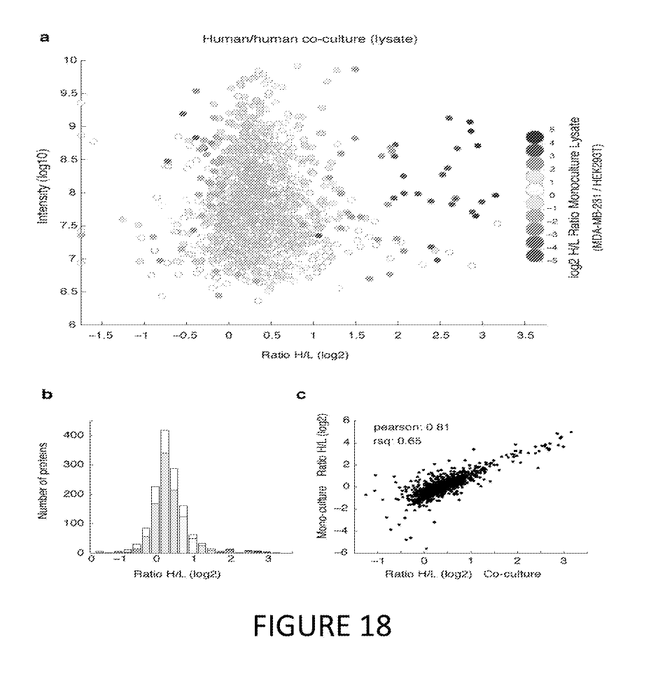

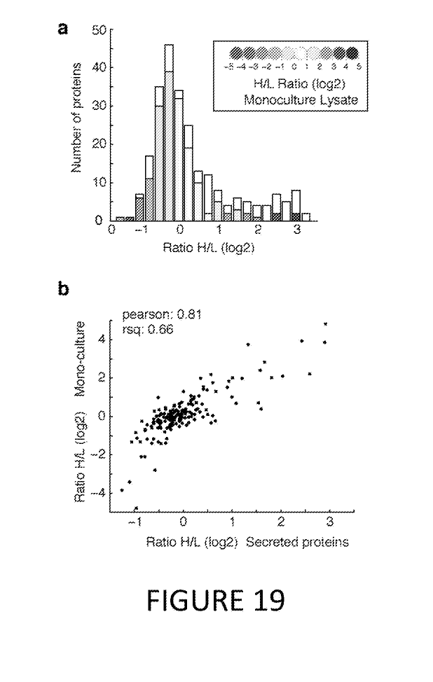

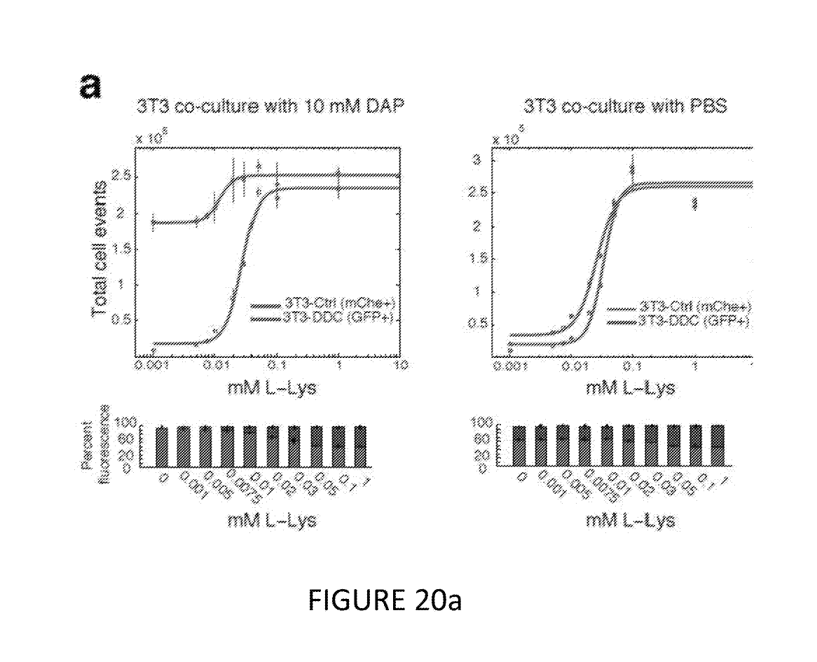

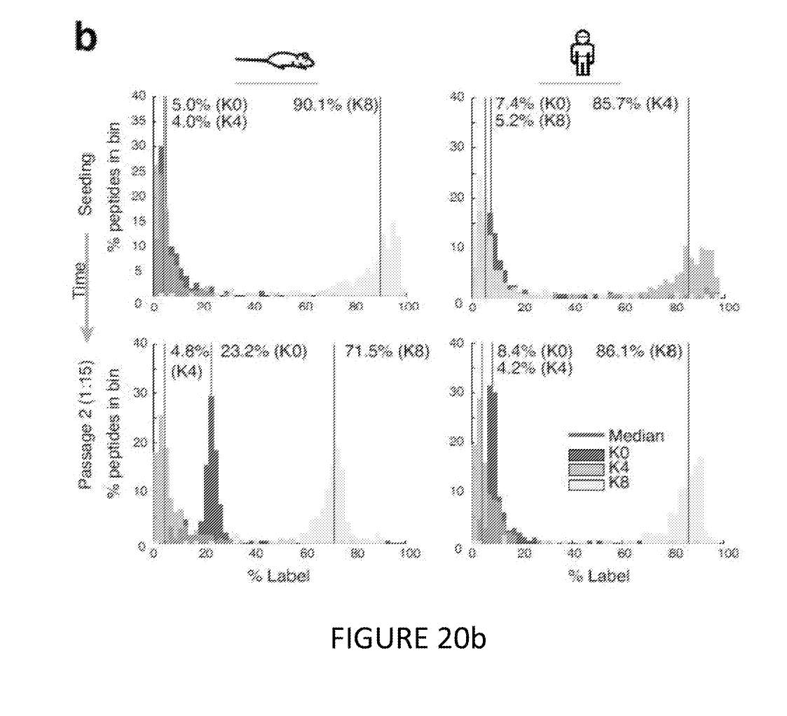

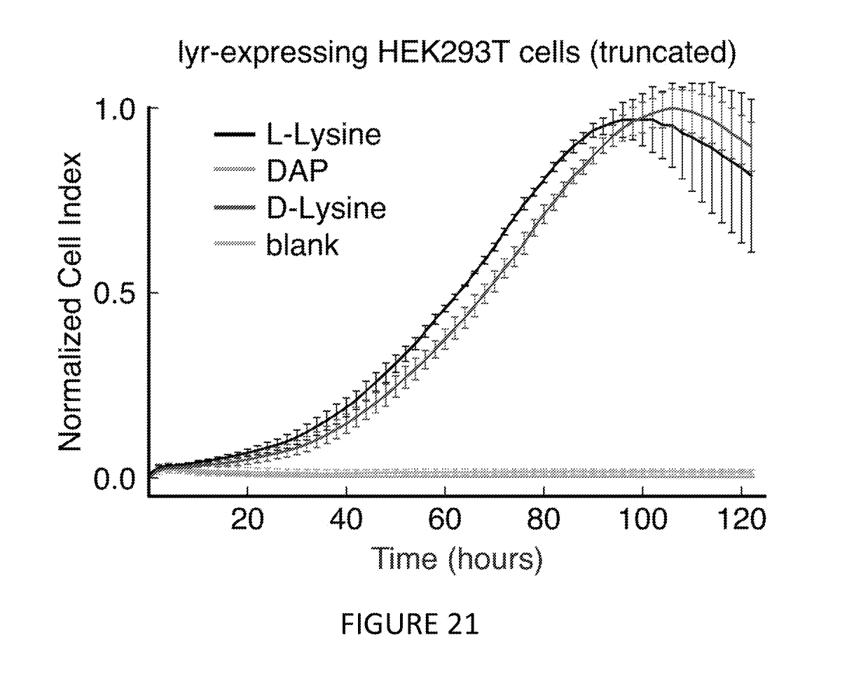

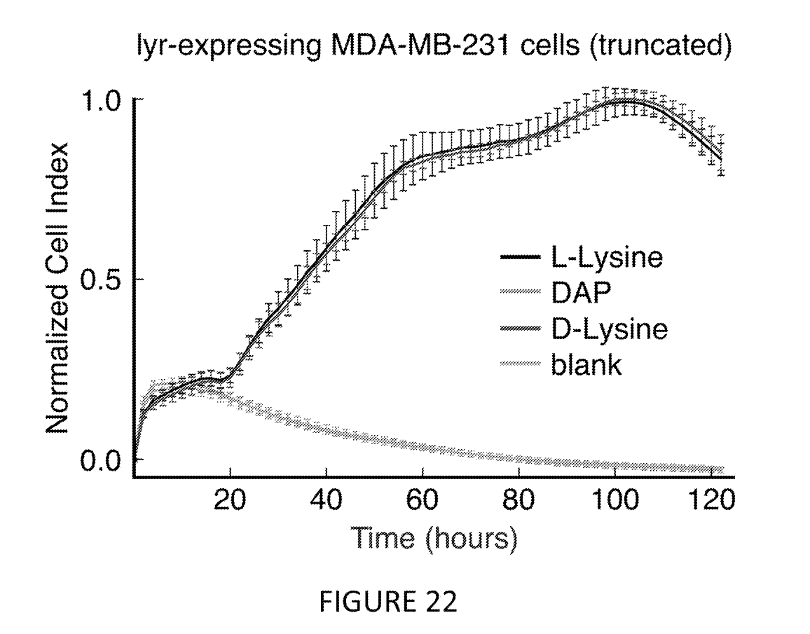

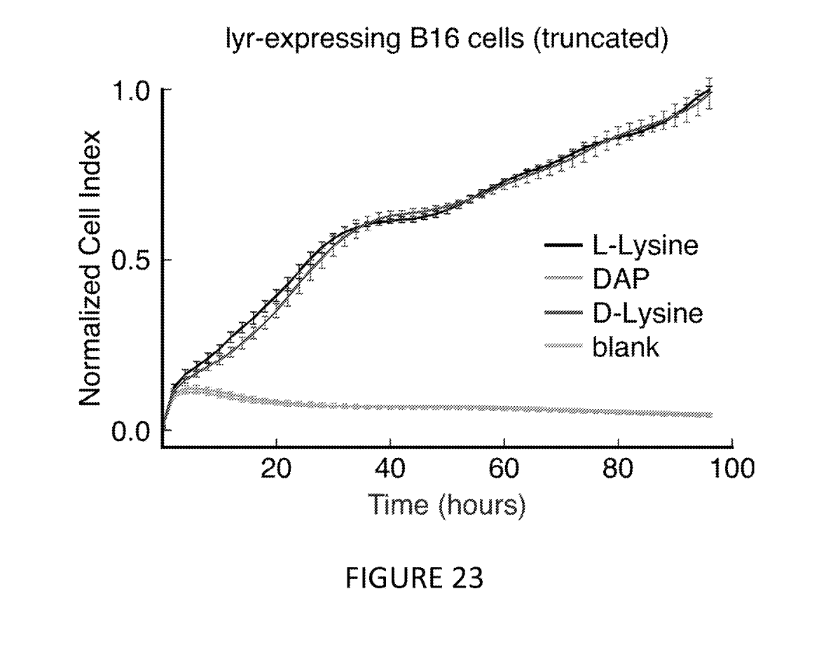

Applying a similar approach for analyzing secreted factors in a same-species co-culture, supernatant was collected and subjected to LC-MS/MS from the same co-cultured DDC-expressing HEK293T and lyr-expressing MDA-MB-231 cells as previously used. Quantitative analysis of the H/L ratios of 245 identified proteins spanned a similar range as those detected intracellularly with the tails of the distribution representing proteins primarily expressed in one cell type (FIG. 6b). Analysis of the human-mouse co-culture showed distinct labeling of secreted proteins and similar labeling specificity is expected for proteins secreted in the same-species co-culture. However, to gain more confidence that the H/L ratios reflect the relative protein abundance between each cell-type, we investigated whether intracellular protein levels correlate with those found extracellularly. Quantitated protein ratios of mixed mono-culture lysates were therefore related to their secreted counterparts. Focusing on the subset of proteins that were common to both samples, good agreement was observed between the H/L ratios from the intracellular mono-culture and secreted co-culture samples (R2=0.66, pearson correlation=0.81, FIG. 6b and FIG. 13). Considering the differences in culture conditions and localization of the harvested proteins, this correlation was surprisingly high. In concordance with the human-mouse secretome analysis, proteins with the lowest and highest H/L ratios are likely secreted from the HEK293T and MDA-MB-231 cells, respectively. As the secretome of MDA-MB-231 cells has been previously investigated and the high ratio proteins were readily separable from the majority of the identified proteins, we focused on the proteins the highest ratios. Indeed, of the top five proteins, three have recently been reported to be secreted by MDA-MB-231 cells (FIG. 6b) (18). Interestingly, a relatively large proportion of these putative MDA-MB-231-secreted proteins could not be identified intracellularly, highlighting the need for secretome profiling. Taken together with the species-verified secretome analysis, these results establish that the CTAP method can be applied to determine the cell-of-origin for differentially label secreted factors in co-culture.

The following are representative applications of the CTAP methodology disclosed herein.

### Identifying and Developing Cancer Therapeutics in Context of Tumor Microenvironment

Microenvironment-mediated drug resistance is understudied and likely plays an important role in the failure of many therapies. For example, studies have implicated bone marrow cells as playing an important role in multiple myeloma resistance to the glucocorticoid, dexamethasone. Response to other drugs, such as DNA intercalating agent doxorubicin, have been less clear, showing enhanced effects in certain tumor-stromal contexts and are attenuated effects in others.

In vitro co-culture models of stroma-tumor interactions have been developed for cancer drug screening, however, these models are largely limited to phenotypic end-points such as cell growth or death. The molecular mechanisms of the cell-cell interactions are under-appreciated, partly due to the fact that current methods are unable to discriminate proteins originating from the different cell types. The CTAP method can facilitate the development of targeted therapies directed at malignant tumor-stroma interactions as well as help understand the mechanisms leading to stromal mediated drug resistance or sensitivity.

### In Vivo Biomarker Discovery

The CTAP methodology is also applicable in vivo as the enzyme can be expressed in a tissue or cell-specific manner in genetically modified animals. A particular cell type of interest is engineered to express the enzyme using cell-specific promoters, and a labeled precursor is administered to the animal, leading to selective labeling of the transgenic cells. Labeled proteins secreted from these cells can be detected in proximal fluids or in the serum and thus serve as unambiguous cell-specific biomarkers. By focusing on proteins that come from diseased tissue, it is more likely that markers that are indicative of disease development, maintenance, or outcome can be found. Current biomarker discovery techniques, which rely solely on statistical methods to prioritize proteins important for diagnosis or prognosis do not have this advantage as they are unable to determine from what cell-type the biomarker originates.

The following methodology is used in practicing the disclosed invention.

### Oligonucleotide Acquisition

The L-Lysine producing enzymes used in this study were DDC, lyr, and CBZcleaver. DDC was directly amplified by PCR from Arabidopsis Thaliana cDNA (TAIR id=AT3G14390, primer sequences shown in Table S1). The lyr and CBZcleaver constructs were synthesized by GeneArt with the amino acid sequence specified by Kuan et al. [22] and Naduri et al. [23] respectively, with nucleotide sequences optimized for expression in mouse. Sequences were verified for all plasmids by the Sanger method of sequencing.

### Plasmid Construction, Virus Production, and Cell Line Generation

Two MSCV based retroviral vector backbones, one expressing GFP (pMIG) and the other mCherry (pMIC), were used to infect mouse cells. For insert into pMIG, the PCR product of DDC was cloned into the EcoRI site of the vector. CBZcleaver was directly subcloned from the GeneArt supplied vector pMA-RQ into pMIC using EcoRI and Xhol restriction sites. Viral supernatants for pMIG and pMIC were produced by transfecting Phoenix cells with each plasmid and the supernatant was used to infect 3T3 cells 48 hours later as previously described [24; 25].

The lentiviral backbone pLM was used to infect human cells. Overlapping PCR was performed to generate eGFP-DDC and mCherry-lyr constructs that were linked by a P2A peptide preceded by a Gly-Ser-Gly linker [26]. The pLM-P2A-enzyme virus was packaged by calcium phosphate transfection of the HEK293T packaging cell line using 10 μg of transfer vector, 6.5 μg of CMV6R8.74, and 3.5 μg of the VSV.G plasmid. MDA-MB-231 and HEK293T cells were then infected with lentiviral supernatant produced from the pLM construct 48 hours post-transfection of the packaging line.

### Cellular Growth Assays

Cell lines were grown in Dulbecco's modified Eagle's medium (DMEM) without L-Lysine and L-Arginine (SILAC-DMEM, Thermo Fisher Scientific) supplemented with 10% dialyzed FBS, antibiotics, and L-glutamine. For mono-culture growth assays, 1 mM L-Arginine was added to the media and cells were seeded in 200 μL in 96-well plates with 4000 or 5000 cells per well in different concentrations of L-Lysine, meso-2,6-diaminopimelate (DAP, Sigma, 33240), D-Lysine HCL (Sigma, L5876), N-α-Cbz-L-Lysine (Z-Lysine, BaChem, C-2200), or N₂-acetyl-L-Lysine (N2A, Sigma, A2010). Cell viability was measured using either the metabolic-activity based Resazurin (Sigma) reagent or the impedance-based xCELLigence system (Roche). For Resazurin experiments, 25 μL of the Resazurin reagent was added to each well and cellular growth was estimated after two to three hours of incubation at 37° C. as described by the manufacturer. For xCELLigence experiments, cells were plated in either 16 or 96-well E-plates, allowed to settle for 30 minutes at room temperature, and then placed in the RTCA DP or RTCA MP analyzer where impedance was measured every 15 minutes for 96-120 hours. At least three replicates were performed for each condition.

Measuring the percentage of mCherry⁺ and GFP⁺ cells in co-culture was performed by either flow cytometry (BD LSR II) or Tali image-based cytometry (Invitrogen). For flow cytometric assays, 25,000 cells from each cell line were seeded together in 6-well plates in 3-4 mL media supplemented with different concentrations of L-Lysine and/or L-Lysine precursors. After 72 hours, cells were trypsinized, washed, and resuspended in 200 μL PBS containing 2% dialysed FBS and 0.1% NaN3. 20 μL was used for estimating total cell numbers using the ViaCount assay (Guava Technologies/Millipore) as described by the manufacturer. The remaining 180 μL was mixed with an equal volume of 2% paraformaldehyde. The percentage of GFP⁺ and mCherry⁺ cells in each sample was analyzed by flow cytometry. At least two replicates were performed for each condition. For Tali assays, cells were trypsinized, resuspended in media, 25 μL of co-culture cell suspension was used to determine the percentage of GFP⁺ and RFP⁺ cells in biological triplicate.

mRNA Microarray Expression Profiling

Cells were seeded at equal densities into SILAC media containing 798 μM K0, 798 μM K4, 4 mM D-Lysine HCl, or 10 mM DAP. After 72 hours, cells were washed, trypsinized, pelleted, and frozen at −80° C. RNA was extracted using the RNeasy mini kit (Qiagen), labeled, and hybridized to Illumina mouseref-8 or Human HT-12 microarrays. After median centering the probe intensities for each array, moderated t-statistics and false discovery rate calculations for multiple hypothesis correction were performed using the eBayes method provided in LIMMA (27; 28).

### Stable Isotope Labeling and Cell Passaging

For exchange-of-label experiments (all monocultures, all human-mouse co-cultures, and Supplementary FIG. S4), cells were first metabolically labeled by growth for at least 10 cellular doublings in SILAC DMEM containing 798 μM light L-Lysine (K0), medium [²H₄]L-Lysine (K4), or heavy [¹³C₆, ¹⁵N₂]L-Lysine (K8) (Cambridge Isotopes). Cells were then seeded in mono- or co-culture with 10 mM light meso-2,6-diaminopimelate (DAP0, Sigma), 2.5 mM or 4 mM heavy [²H₈]D-Lysine (DLYS8, C/D/N Isotopes), 2.5 mM heavy labeled [¹³C₆, ¹⁵N₂]Z-Lysine (Z8, Figure S4), or both DAP0 and DLYS8. For experiments that maintained label (all human-human co-cultures), cells were initially grown for at least 10 cellular doublings in their respective precursors (DDC-expressing in DAP0, lyr-expressing in DLYS8). Populations were then combined in 10 mM DAP and 3 mM DLYS8 and grown together for 5 days in co-culture (approximately 4 cellular doublings). All cell lines were passaged 1:10-1:15 at 95% confluence.

### Mass Spectrometry Sample Preparation

For cultured media samples, cells were washed three times with PBS and supplied with serum-free SILAC DMEM 24 hours prior to supernatant sample collection. Media was collected, filtered with a 0.22 μm filter, and proteins were concentrated to around 1 mg/mL using a 3 KDa Amicon Ultra Centrifuge filter (Millipore) as described by the manufacturer. For harvesting of cell lysate, cells were trypsinized, resuspended in SILAC DMEM, washed three times in ice cold PBS, and cell pellets frozen at −80° C. For FACS samples, co-cultures of GFP+ and mCherry⁺ cells were trypsinized, washed, and resuspended in PBS with 20% media (2% FBS) to a concentration of approximately 2×107 cells/mL. Cells were then sorted into single GFP+ and mCherry⁺ populations on a MoFlo cell sorter (Dako), washed twice with ice cold PBS, and cell pellets were stored at −80° C. for further analysis.

### Protein Extraction/Digestion

Cell pellets were resuspended with Denaturation buffer (6 M Urea/2 M thio Urea in 10 mM Tris), 1 μL of benzonase was added, followed by incubation for 10 minutes at room temperature. Cellular debris was removed by centrifugation at 4000 g for 30 min. For the supernatant samples, the secreted proteins were precipitated by chloroform/methanol extraction. Protein concentration was assessed by the Bradford assay (Bio-Rad). Crude protein extracts were subjected to either GelC or in-solution digest. For the GeLC-MS analysis, protein extracts were cleaned on a 10 cm, 4-12% gradient SDS-PAGE gel (Novex). The resulting lane was cut from the gel and subjected to in-gel digestion with trypsin as described previously (29). Upon gel extraction, peptides were cleaned using Stage-tips and analyzed by nano-LC-MS. For in-solution digestion, proteins from the crude extract were reduced with 1 mM dithiothreitol (DTT), alkylated with 5 mM iodoacetamide, predigested with the endoproteinase Lys-C (Wako) for 3 h, and further digested with trypsin overnight (30). The resulting peptide mixture was cleaned using Stage-tips (31) and subjected to nano-LC-MS without prior peptide separation.

### LC-MS/MS Analysis

All samples were analyzed by online nanoflow liquid chromatography tandem mass spectrometry (LC-MS/MS) as previously described (32) with a few modifications. Briefly, nanoLC-MS/MSexperiments were performed on an EASY-nLC™ system (Proxeon Biosystems, Odense, Denmark) connected to an LTQ-Orbitrap XL or LTQ-Orbitrap Elite (Thermo Scientific, Bremen, Germany) through a nanoelectrospray ion source. Peptides were auto-sampled directly onto the 15 cm long 75 mm-inner diameter analytical column packed with reversed-phase C18 Reprosil AQUA-Pur 3 mm particles at a flow rate of 500 nl/min. The flow rate was reduced to 250 nl/min after loading, and the peptides were separated with a linear gradient of acetonitrile from 545% in 0.5% acetic acid for either 100, 150, or 240 minutes. Eluted peptides from the column were directly electrosprayed into the mass spectrometer. For the LTQ-Orbitrap XL analyses, the machine was operated in positive ion mode, with the following acquisition cycle: a full scan recorded in the orbitrap analyzer at resolution R 120,000 was followed by MS/MS (CID) of the top 10 most intense peptide ions in the LTQ analyzer. The total acquisition gradient was either 150 or 240 minutes. For LTQ-Orbitrap Elite data acquisition the machine was operated in the positive ion mode, with the following acquisition cycle: a full scan recorded in the orbitrap analyzer at reso-lution R 120,000 was followed by MS/MS (CID Rapid Scan Rate) of the 20 most intense peptide ions in the LTQ analyzer. The total acquisition gradient was either 100 or 240 minutes depending on the method of sample preparation. Mono-enzyme co-culture samples were measured on the LTQ-Orbitrap XL with slight modifications: a full scan recorded in the orbitrap analyzer at resolution R 120,000 was followed by MS/MS (CID) of the top 5 most intense peptide ions, with a total acquisition gradient of 95 minutes.

### Processing of MS Data

The MaxQuant software package (version 1.2.2.9) with the Andromeda search engine was used to identify and quantify proteins in cellular lysates and media (14; 33). Mouse and human IPI protein databases (both version 3.84, http://www.ebi.ac.uk/IPI/) plus common contaminants were used. With the exception of “second peptides”, which was deselected, default parameters were selected. For L-Lysine derived from precursors DAP, Z8, and DLYS8, variable labels were specified as K0, K8, and a custom modification (8 deuterium atoms for L-Lysine), respectively. Detection of non-precursor-based L-Lysine was specified as K0, K4, and K8.

Peptide and protein statistics (e.g., sequences, H/L ratios, intensities) were extracted from MaxQuant exported peptides.txt and proteingroups.txt, respectively. Peptides were determined to be species-specific if they only appeared in either one of the human or mouse IPI protein databases. Percent heavy label was calculated from the H/L ratio (HtoL) as =100*HtoL/(HtoL+1). In order to determine the overlap of H/L ratios between the human and mouse sequence-specific peptides, the median H/L ratio of each species was first determined. Next, the average of these two median values was used as a separator for each cell type and the miscategorizations were determined by the percentage of misclassified peptides on either side of this separator.

### Data

The raw data associated with this study will be released upon manuscript acceptance.

### Drug Perturbation Assay

Cells were seeded in 96-well plates (2000 cells/well) and grown to 40% confluence in SILAC media containing 798 μM Ko or 10 mM DAP DMEM with 10% dialyzed fetal bovine serum (FBS). Cells were then inhibited with eight different drug concentrations (2 fold dilution) in eight replicates. Drugs used were Stattic (STAT3 inhibitor), PI3K-IV (PI3K inhibitor), AKT-VIII (AKT inhibitor), and SL327 (MEK inhibitor). After 48 hours drug treatment cell viability was measured by Resazurin (Sigma) as described by manufacturer. Cell viability relative to untreated cells was calculated to obtain dose-response curves.

### Western Blotting

Frozen cell pellets were thawed and lysed for 20 minutes with NP40 lysis buffer, which contained 1% Nonidet P-40, 1 mM sodium orthovanadate, and Complete protease inhibitors (Roche Diagnostics) in PBS. Protein concentrations were determined by the Bradford assay (BioRad) and adjusted to 1-1.5 mg/mL. Protein was then denatured in 2% SDS for 5 minutes at 95° C. Approximately 20 μg of each sample was then separated by SDS-PAGE, transferred to PVDF membrane, and immunoblotted using primary and secondary antibodies. All antibodies were from Cell Signaling. Chemoluminescence visualization was performed on Kodak or HyBlotCL films and films were scanned by a microTEK scanner at 600 d.p.i. in gray scale. The membranes were stripped and reprobed with anti-GAPDH (Cell Signaling) to test for protein loading.

Synthesis of Z-Lysine [N^{α}-Cbz-lysine (K8)]

To a solution of saturated aqueous NaHCO₃ (1.25 mL) and L-lysine.2HCl (250 mg, 1.11 mmol, 1.00 equiv) was added solid NaHCO₃ (105 mg, 1.13 equiv, 1.25 mmol) followed by aqueous CuSO₄ (1.5 mL, 0.50 M, 0.68 mmol 0.60 equiv), immediately forming a blue copper complex. After stirring for 10 min, di-tert-butyl dicarbonate (325 mg, 1.49 mmol, 1.35 equiv) was added in 1 mL acetone. After stirring for 16 h, additional di-tert-butyl dicarbonate solid (150 mg, 0.621 equiv, 0.690 mmol) was added. After 24 h, the reaction was quenched with methanol (1 mL) and stirred for an additional 16 h. Ethyl acetate (1 mL) and water (1 mL) were added and the heterogeneous suspension was filtered. The recovered blue solid was taken up in H₂O (3 mL), sonicated for 30 s, and filtered. After air drying, the N^{ε}-Boc-protected copper complex was collected as a fine periwinkle blue powder (235 mg, 0.423 mmol, 74.2% yield), which was used without further purification.

To a suspension of N^{ε}-Boc-protected copper complex (235 mg, 0.417 mmol, 1.00 equiv) in acetone (1.5 mL) was added 8-hydroxyquinoline (130 mg, 0.900 mmol, 2.13 equiv) and 10% Na₂CO₃ (1.8 mL). After 1 h, N-(Benzyloxycarbonyloxy)succinimide (205 mg, 0.821 mmol, 1.97 equiv) in 1 mL acetone was added dropwise over 10 min and stirred for 1 h. The reaction mixture was filtered, and the residue washed with water (3×1 mL). The pale green filtrate was acidified carefully with 1 N HCl to a pH of 2, and extracted with ethyl acetate (2×5 mL). The combined organics were washed with brine, dried over sodium sulfate, filtered, and concentrated by rotary evaporation to afford crude N^{ε}-Boc-N^{α}-Cbz-L-lysine(K8) (148 mg, 45.7% yield, 0.381 mmol).

To a solution of crude N^{ε}-Boc-N^{α}-Cbz-L-lysine(K8) (148 mg, 0.381 mmol, 1.00 equiv) in acetone (1.7 mL) was added T_{S}OH.H₂O (145 mg, 0.762 mmol, 2.00 equiv). After 16 h, crystals were collected by vacuum filtration and washed sparingly with cold acetone, giving N^{α}-Cbz-lysine(K8).T_{S}OH (124 mg, 71.0% yield, 0.270 mmol).

Crude N^{α}-Cbz-lysine(K8).T_{S}OH was dissolved in 1.0 mL 5% acetonitrile (v/v in water), treated with triethylamine (37.5 μL, 0.269 μmol, 1.00 equiv), and purified on a 5.5 g C-18 ISCO RediSep Gold column (5→90% acetonitrile in H2O). Lyophilization furnished N^{α}-Cbz-lysine(K8) as a fluffy white amorphous solid (77 mg, 0.27 mmol, 99% yield).

¹H NMR (D₂O, 600 MHz) (δ 7.25-7.35 (m, 5H), 5.04 (d, J=12.5 Hz, 1H), 4.97 (d, J=12.5 Hz, 1H), 3.83 (dm, J_{CH}=140.4 Hz, 1H), 2.84 (dm, J_{CH}=142.8 Hz, 2H), 1.66 (dm, J_{CH}=128.4 Hz, 1H), 1.53 (dm, J_{CH}=131.4 Hz, 3H), 1.28 (dm, J_{CH}=132.6 Hz, 2H); ¹³C-NMR (D₂O, 151 MHz) δ 179.8 (d, J=8.4 Hz), 179.5 (d, J=8.4 Hz), 128.7 (s), 128.2 (s), 127.6 (s), 66.8 (s), 56.2 (ddd, J=138.0, 46.2, 14.4 Hz), 55.8 (ddd, J=139.2, 46.8, 15.0 Hz), 34.2 (dt, J=161.0, 18.6 Hz), 31.1 (td, J=

138.6, 18.0 Hz), 23.2 (td, J=138.6, 18.8 Hz), 22.0 (t, J=137.4 Hz); [α]¹⁹_{D}: −12.50±0.04° (c=2.00, 0.2 N HCl); FTIR (solid, cm⁻¹) 3306, 3031, 2931, 1717, 1654, 1497, 1402, 1369, 1344, 1232; ESI-HRMS (m/z): calcd for C₈ ¹³C₆H₂₁ ¹⁵N₂O₄ (M+H)⁺289.1643. found 289.1650.

### REFERENCES

[1] Gygi, S. P. et al. Quantitative analysis of complex protein mixtures using isotope-coded affinity tags. Nat Biotechnol 17(10), 994-9 October (1999).
[2] Ong, S.-E. et al. Stable isotope labeling by amino acids in cell culture, silac, as a simple and accurate approach to expression proteomics. Molecular & Cellular Proteomics 1(5), 376-86 May (2002).
[3] Ross, P. L. et al. Multiplexed protein quantitation in saccharomyces cerevisiae using amine-reactive isobaric tagging reagents. Molecular & Cellular Proteomics 3(12), 1154-69 December (2004).
[4] Jørgensen, C. et al. Cell-specific information processing in segregating populations of eph receptor ephrin-expressing cells. Science 326(5959), 1502-9 December (2009).
[5] Rechavi, O. et al. Trans-silac: sorting out the non-cell-autonomous proteome. Nat Methods 7(11), 923-7 November (2010).
[6] Naba, A. et al. The matrisome: in silico definition and in vivo characterization by proteomics of normal and tumor extracellular matrices. Mol Cell Proteomics 11(4), M111.014647 April (2012).
[7] van den Bemd, G.-J. C. M. et al. Mass spectrometric identification of human prostate cancer-derived proteins in serum of xenograft-bearing mice. Mol Cell Proteomics 5(10), 1830-9 October (2006).
[8] Ngo, J. T. et al. Cell-selective metabolic labeling of proteins. Nat Chem Biol 5(10), 715-7 October (2009).
[9] Truong, F., Yoo, T. H., Lampo, T. J. & Tirrell, D. A. Two-strain, cell-selective protein labeling in mixed bacterial cultures. Journal of the American Chemical Society May (2012).
[10] Liu, C. C. & Schultz, P. G. Adding new chemistries to the genetic code. Annual review of biochemistry 79, 413-44 January (2010).
[11] Xu, H., Andi, B., Qian, J., West, A. H. & Cook, P. F. The alpha-aminoadipate pathway for lysine biosynthesis in fungi. Cell Biochem Biophys 46(1), 43-64 January (2006).
[12] Saqib, K. M., Hay, S. M. & Rees, W. D. The expression of escherichia coli diaminopimelate decarboxylase in mouse 3t3 cells. Biochim Biophys Acta 1219(2), 398-404 October (1994).
[13] Jouanneau, J., Stragier, P., Bouvier, J., Patte, J. C. & Yaniv, M. Expression in mammalian cells of the diaminopimelic acid decarboxylase of escherichia coli permits cell growth in lysine-free medium. Eur J Biochem 146(1), 173-8 January (1985).
[14] Cox, J. & Mann, M. Maxquant enables high peptide identification rates, individualized p.p.b.-range mass accuracies and proteome-wide protein quantification. Nat Biotechnol November (2008).
[15] Sury, M. D., Chen, J.-X. & Selbach, M. The silac fly allows for accurate protein quantification in vivo. Mol Cell Proteomics 9(10), 2173-83 October (2010).
[16] Spellman, D. S., Deinhardt, K., Darie, C. C., Chao, M. V. & Neubert, T. A. Stable isotopic labeling by amino acids in cultured primary neurons: application to brain-derived neurotrophic factor-dependent phosphotyrosine-associated signaling. Mol Cell Proteomics 7(6), 1067-76 June (2008).
[17] Geiger, T., Wehner, A., Schaab, C., Cox, J. & Mann, M. Comparative proteomic analysis of eleven common cell lines reveals ubiquitous but varying expression of most proteins. Mol Cell Proteomics 11(3), M111.014050 March (2012).
[18] Zhang, Y. et al. Lectin capture strategy for effective analysis of cell secretome. Proteomics 12(1), 32-6 January (2012).
[19] Joyce, J. A. & Pollard, J. W. Microenvironmental regulation of metastasis. Nat Rev Cancer 9(4), 239-52 April (2009).
[20] Mcmillin, D. W. et al. Tumor cell-specific bioluminescence platform to identify stroma-induced changes to anticancer drug activity. Nat Med 16(4), 483-9 April (2010).
[21] Olumi, A. F. et al. Carcinoma-associated fibroblasts direct tumor progression of initiated human prostatic epithelium. Cancer Res 59(19), 5002-11 October (1999).
[22] Kuan, Y. et al. Biochemical characterization of a novel lysine racemase from proteus mirabilis bcrc10725. Process Biochemistry (2011).
[23] Nanduri, V., Goldberg, S., Johnston, R. & Patel, R. Cloning and expression of a novel enantioselective n-carbobenzyloxy-cleaving enzyme. Enzyme and Microbial Technology 34(3-4), 304-312 (2004).
[24] Mavrakis, K. J. et al. Tumorigenic activity and therapeutic inhibition of rheb gtpase. Genes & Development 22(16), 2178-88 August (2008).
[25] Swift, S., Lorens, J., Achacoso, P. & Nolan, G. P. Rapid production of retroviruses for efficient gene delivery to mammalian cells using 293t cell-based systems. Curr Protoc Immunol Chapter 10, Unit 10.17C May (2001).
[26] Szymczak, A. L. et al. Correction of multi-gene deficiency in vivo using a single ‘selfcleaving’ 2a peptide-based retroviral vector. Nat Biotechnol 22(5), 589-94 May (2004).
[27] Smyth, G. Limma: linear models for microarray data. Bioinformatics and computational biology solutions using R and Bioconductor, 397-420 (2005).
[28] Benjamini, Y. & Hochberg, Y. Controlling the false discovery rate: a practical and powerful approach to multiple testing. Journal of the Royal Statistical Society. Series B (Methodological) 57(1), 289-300 (1995).
[29] Grgurevic, L., Macek, B., Durdevic, D. & Vukicevic, S. Detection of bone and cartilage-related proteins in plasma of patients with a bone fracture using liquid chromatography-mass spectrometry. International Orthopaedics (SICO 31(6), 743-51 December (2007).
[30] Macek, B. et al. Phosphorylation of the human full-length protein kinase ciota. J Proteome Res 7(7), 2928-35 July (2008).
[31] Ishihama, Y., Rappsilber, J. & Mann, M. Modular stop and go extraction tips with stacked disks for parallel and multidimensional peptide fractionation in proteomics. J Proteome Res 5(4), 988-94 April (2006).
[32] Olsen, J. V. et al. Global, in vivo, and site-specific phosphorylation dynamics in signaling networks. Cell 127(3), 635-48 November (2006).
[33] Cox, J. et al. Andromeda: a peptide search engine integrated into the maxquant environment. J Proteome Res 10(4), 1794-805 April (2011).

1. A method for labeling proteins in a vertebrate cell, the method comprising:
exposing a transgenic vertebrate cell, that expresses an exogenous enzyme that enables the cell to generate an essential amino acid from an essential amino acid substrate/precursor, to a composition comprising said amino acid substrate/precursor for a period of time sufficient for protein synthesis to occur, wherein said essential amino acid in said amino acid substrate/precursor comprises a stable isotope so that proteins synthesized by said transgenic vertebrate cell contain a stable isotope-labeled essential amino acid derived from the substrate/precursor.

2. A method for monitoring protein synthesis in a vertebrate cell, the method comprising:
(a) exposing a transgenic vertebrate cell that expresses an exogenous enzyme that enables the cell to generate an essential amino acid from an essential amino acid substrate/precursor to said amino acid substrate/precursor for a period of time sufficient for protein synthesis to occur, wherein said amino acid substrate/precursor comprises a stable isotope so that proteins synthesized by said transgenic vertebrate cell contain a stable isotope-labeled essential amino acid derived from the substrate/precursor;
(b) isolating proteins from the cell; and
(c) quantifying those proteins containing the stable isotope.

3. A method for differentiating proteins from different cells in a mixed population of vertebrate cells, the method comprising:
(a) exposing
(i) a first transgenic vertebrate cell that expresses an exogenous enzyme capable of converting a precursor/substrate for an essential amino acid to the essential amino acid; and
(ii) a second vertebrate cell
in a mixed population of cells to the essential amino acid and said precursor/substrate for said essential amino acid, wherein one of said essential amino acid and said precursor/substrate is labeled with a stable isotope and the other is unlabeled or labeled with a different stable isotope, for a period of time sufficient for protein synthesis to occur;
(b) recovering proteins from said first and second vertebrate cells;
(c) determining the amount of stable isotope in said proteins to determine cell of origin, wherein the amount of stable isotope indicates whether the protein was synthesized by said first transgenic vertebrate cell or said second vertebrate cell.

4. The method of claim 3, wherein said first and second vertebrate cells are in proximity to each other.

5. The method of claim 3, wherein said first and second cells are exposed in vivo.

6. A method for differentiating proteins from mixed populations of vertebrate cells, the method comprising:
(a) co-culturing
(i) a first transgenic vertebrate cell that expresses a first exogenous enzyme capable of converting a first precursor to an essential amino acid; and
(ii) a second transgenic vertebrate cell that expresses a second exogenous enzyme capable of converting a second precursor to an essential amino acid
in a culture medium comprising said first and second precursors, wherein the essential amino acid in said first precursor comprises a first stable isotope, and the essential amino acid in said second precursor is unlabeled or comprises a second stable isotope for a period of time sufficient for protein synthesis to occur;
(b) recovering proteins from said co-cultured cells;
(c) determining the relative abundance of each of said first and second stable isotopes in said proteins to determine cell of origin, wherein a protein containing said first stable isotope was synthesized by said first transgenic vertebrate cell and a protein comprising no label or said second stable isotope was synthesized by said second vertebrate cell.

7. The method of claim 1, wherein said essential amino acid is lysine.

8. The method of claim 1, wherein said essential amino acid substrate/precursor is selected from the group consisting of labeled or unlabeled meso-2,6-diaminopimelate (DAP), labeled or unlabeled D-Lysine, labeled or unlabeled Z-Lysine.

9. The method of claim 1, wherein said vertebrate cell is transiently or stably transfected to express the exogenous enzyme that produces the essential amino acid from the essential amino acid substrate/precursor.

10. The method of any of the above claim 1, wherein said vertebrate cell is in a transgenic animal.

11. The method of claim 1, wherein said first and second vertebrate cells are mammalian cells.

12. The method of claim 1, wherein said exogenous enzyme is a lysine racemase and the substrate/precursor is D-lysine.

13. The method of claim 1, wherein said exogenous enzyme is a diaminopimelate decarboxylase (DDC) and the substrate/precursor is diaminopimelate (DAP).

14. The method of claim 1, wherein said exogenous enzyme is a CBZcleaver and the substrate/precursor is Z-lysine.

15. (canceled)

16. The method of claim 1, wherein said essential amino acid is lysine.

17. The method of claim 2, wherein the proteins are evaluated by mass spectroscopy.

18. A kit comprising:
(a) a vector for the transfection of vertebrate cells so that the cells express an exogenous enzyme that generates an essential amino acid from an essential amino acid substrate/precursor; and
(b) an unlabeled or a stable isotopically-labeled essential amino acid substrate/precursor.

19. The kit of claim 18, further comprising:
(c) a second vector for the transfection of vertebrate cells so that the cells express a second exogenous enzyme that generates an essential amino acid from an second essential amino acid substrate/precursor; and
(d) an unlabeled or a stable isotopically-labeled second essential amino acid substrate/precursor.

20. The kit of claim 18, wherein said vector comprises a nucleic acid that encodes an enzyme selected from lysine racemase, CBZcleaver and diaminopimelate decarboxylase (DDC).

21. The kit of claim 20, wherein said stable isotopically-labeled essential amino acid substrate/precursor is selected from D-lysine when the enzyme is lysine racemase, Z-lysine when the enzyme is CBZcleaver, and diaminopimelate (DAP) when the enzyme is DDC.

22. The kit of claim 21, further comprising an unlabeled essential amino acid substrate/precursor selected from D-lysine, Z-lysine, and diaminopimelate (DAP).

23. A transgenic cell comprising an exogenous nucleic acid that encodes an enzyme selected from lysine racemase, CBZcleaver and diaminopimelate decarboxylase (DDC).

24. A transgenic non-human animal comprising a exogenous nucleic acid that encodes an enzyme selected from lysine racemase, CBZcleaver and diaminopimelate decarboxylase (DDC).

25. The transgenic non-human animal of claim 24, wherein said animal comprises first and second exogenous nucleic acids each of which encode a different enzyme selected from lysine racemase, CBZcleaver and diaminopimelate decarboxylase (DDC).

26. The transgenic animal of claim 25, wherein said first and second exogenous nucleic acids are expressed in different cell types.

27. The method of claim 1, wherein said cell is exposed to a composition comprising said amino acid substrate/precursor but lacking said essential amino acid.


SEQUENCE LISTING (14 sequences)

SEQ ID NO: 1 - DNA, 28 bp, Artificial Sequence
  Synthetic oligonucleotide
gccctcgagatggcggcagctactcaat

SEQ ID NO: 2 - DNA, 33 bp, Artificial Sequence
  Synthetic oligonucleotide
cgcgaattcgttcatagaccttcaaagaaacgc

SEQ ID NO: 3 - DNA, 28 bp, Artificial Sequence
  Synthetic Oligonucleotide
atcgaattcatggcggcagctactcaat

SEQ ID NO: 4 - DNA, 33 bp, Artificial Sequence
  Synthetic Oligonucleotide
ccggttaccggtatggtgagcaagggcgaggag

SEQ ID NO: 5 - DNA, 88 bp, Artificial Sequence
  Synthetic Oligonucleotide
agggccgggattctcctccacgtcacctgcttgtttgagtagtgagaagtttgttgctccagatcccttgtacagctcgtccatgccg

SEQ ID NO: 6 - DNA, 85 bp, Artificial Sequence
  Synthetic Oligonucleotide
ggatctggagcaacaaacttctcactactcaaacaagcaggtgacgtggaggagaatcccggccctatggcggcagctactcaat

SEQ ID NO: 7 - DNA, 35 bp, Artificial Sequence
  Synthetic Oligonucleotide
ccggttgtcgactcatagaccttcaaagaaacgca

SEQ ID NO: 8 - DNA, 87 bp, Artificial Sequence
  Synthetic Oligonucleotide
ggatctggagcaacaaacttctcactactcaaacaagcaggtgacgtggaggagaatcccggccctatgagcctgggcatcagatac

SEQ ID NO: 9 - DNA, 28 bp, Artificial Sequence
  Synthetic Oligonucleotidde
tgttgtcgactcaatccaccagcacgcg

SEQ ID NO: 10 - DNA, 1455 bp, Arabidopsis thaliana
atggcggcagctactcaatttctctcccaaccttcgtctctcaatccacaccaactgaagaaccaaacctcacaacgctccagaagcatccctgtcttgtctcttaaatccacattgaagccacttaaacgcctctccgtgaaagccgccgtcgtttctcaaaactcgtccaaaaccgtgacgaagttcgatcactgtttcaagaaatcatcagatgggtttctctattgtgaaggaactaaagttgaggatatcatggagtcagtggagagaagacccttttacttatatagcaaacctcagatcactagaaacctcgaggcttataaagaagcattggaaggagtgagctctgtgattggttacgctatcaaagctaataacaatcttaaaattttggagcatttgagaagtttaggctgtggtgctgtgctcgttagtggaaatgagcttagacttgctcttcgtgctggtttcgatcccacaaagtgcattttcaatggaaatggcaagtctttggaagatttagttctagctgctcaagaaggtgttttcgttaatgtcgatagtgagtttgacttgaataacattgtggaagcttcaagaatttctggtaagcaggtcaatgtactgctgcgtatcaatcctgatgttgatcctcaggtgcatccatatgttgctactgggaacaagaactcaaagtttggtatcaggaacgagaagcttcaatggtttctggatcaggtcaaggcacatcccaaagagctgaagcttgttggagctcattgccatctaggctctaccattactaaggtggatatattcagagatgcggcagttctcatgatagaatacattgacgagatccggcgtcaaggttttgaagttagttacttgaacattggtggtggtttagggattgattattaccatgccggcgctgtccttcccacacccatggatctcatcaacactgtaagagagcttgttctttcacgagacctgaatctaataatcgagccagggagatctctgattgcaaacacttgctgtttcgtcaaccatgtaactggtgtgaagacgaatggaactaagaacttcatagtcattgatggaagtatggctgagcttatccgtcccagtctttatgatgcttatcagcacattgagttggtctctcctccaccggctgaagcagaggttaccaaattcgacgtagtgggtcctgtctgtgaatctgctgatttcctgggcaaagacagagagcttcccactcctccacagggagctggtctggtggttcatgacgctggtgcatactgtatgagcatggcttccacttacaatctcaagatgcgtcctccggaatactgggttgaagaagatgggtcgatcactaagataaggcatgctgagacattcgatgaccatttgcgtttctttgaaggtctatga

SEQ ID NO: 11 - DNA, 1224 bp, Proteus mirabilis
atgagcctgggcatcagatacctggccctgctgcccctgttcgtgatcaccgcttgtcagcagcccgtgaactacaacccccctgccacacaggtggcccaggtgcagcctgccatcgtgaacaacagctggatcgagatcagcagaagcgccctggacttcaacgtgaagaaggtgcagagcctgctgggcaagcagagcagcctgtgtgctgtgctgaagggcgacgcctacggccacgatctgtctctggtggcccccatcatgatcgagaacaatgtgaagtgcatcggcgtgaccaacaaccaggaactgaaagaagtgcgggacctgggcttcaagggcagactgatgagagtgcggaacgccaccgagcaggaaatggcccaggccaccaactacaacgtggaagaactgatcggcgacctggacatggccaagagactggacgctatcgccaagcagcagaacaaagtgatccccatccacctggctctgaacagcggcggcatgagcagaaacggcctggaagtggacaacaagtctggcctggaaaaggccaagcagatctcccagctggccaacctgaaggtcgtgggcatcatgagccactaccccgaagaggacgccaacaaagtgcgcgaggacctggcccggtttaagcagcagtctcagcaggtgctggaagtgatgggcctggaacggaacaacgtgaccctgcacatggctaacaccttcgccaccatcaccgtgcccgagagctggctggatatggtgcgagtgggcggcatcttctacggcgacacaatcgccagcaccgactacaagagagtgatgaccttcaagagcaatatcgcctccatcaactactaccccaagggcaacaccgtgggctacgacagaacctacaccctgaagagggacagcgtgctggctaacatccccgtgggatacgccgacggctacagaagagtgttcagcaacgccggccacgccctgatcgctggacagagggtgccagtgctgggaaagaccagcatgaacaccgtgatcgtggacatcaccagcctgaacaacatcaagcccggcgacgaggtggtgttcttcggcaagcagggcaacagcgagatcaccgccgaggaaatcgaggacatctctggcgccctgttcaccgagatgagcatcctgtggggcgccaccaatcagcgcgtgctggtggattga

SEQ ID NO: 12 - DNA, 1281 bp, Sphingomonas paucimobilis
atggtgcagcccacccctacccctcagagcgagctgcctggcctgatcgccagagacatggaaggcctgatgacactgtacagggacctgcacgccaaccccgagctgagcctgcaggaagtgaacaccgccgccaagctggccaagagactgaaggccatgaagttcgacgtgacagagaaagtgggcggcaccggcgtggtggccgtgatgaagaacggcagcggacctgtgctgctgatcagagccgatatggacggcctgcccgtggtggaacagaccggcctggacttcgccagcaaagtgcggaccaagacccccgagggcgtggaaacaggcgtgatgcacgcttgcggccacgacacccacatgaccgccttcatcgagacagccaagctgctgagcagccagaaagacaagtggaagggcaccctggtgatgatcctgcagcccgccgaggaagtgggcaagggcgccagggacatgctggaagatggcctgtacaccagattccccagacccacccacgctatcgccttccacgacgccgccaacctccaggctggcgtggtgggatacacccctggctacgccctggccaacgtggacagcgtggacatcgtggtgaaaggcctgggaggacacggcgcctacccccagaccaccagagatcccatcgtgctgggcagcagaatcgtgaccagcctgcagaccctggtgtccagagagcaggacccccaggaccctgccgtggtgacagtgggctctttccaggctggcgccaagcacaacatcatccccgaccaggctctgctgctgctgaccgtgcggagctacagcgacgagacaagagccaagctgatcaagggcatcgagagaatcgccagaggcgaggctatcgccgctggcgtgcccgacgacaagatgcctgtggtgtccgtgaaggacgagttcacccccagcacctacaacccccccgagttcgccgagcagatgggcgctctgctgaagggacacttcgccgagggcagagtggtgaaaacccctgccgtgatgggcggcgaggacttcggcagattctacagagccgacaagtctatcaacagcttcatcttctgggtgggaggcgtgccagccgataagatggccgctgcccaggccggccagatcacactgcctagcctgcacagccctttctgggcccctgaggccgacaaagtgatcgccaccgccagcgaggccatgaccgtgctggccatggacatcctgaagaaggactga

SEQ ID NO: 13 - DNA, 1188 bp, Proteus mirabilis
atgcagcagcccgtgaactacaacccccctgccacacaggtggcccaggtgcagcctgccatcgtgaacaacagctggatcgagatcagcagaagcgccctggacttcaacgtgaagaaggtgcagagcctgctgggcaagcagagcagcctgtgtgctgtgctgaagggcgacgcctacggccacgatctgtctctggtggcccccatcatgatcgagaacaatgtgaagtgcatcggcgtgaccaacaaccaggaactgaaagaagtgcgggacctgggcttcaagggcagactgatgagagtgcggaacgccaccgagcaggaaatggcccaggccaccaactacaacgtggaagaactgatcggcgacctggacatggccaagagactggacgctatcgccaagcagcagaacaaagtgatccccatccacctggctctgaacagcggcggcatgagcagaaacggcctggaagtggacaacaagtctggcctggaaaaggccaagcagatctcccagctggccaacctgaaggtcgtgggcatcatgagccactaccccgaagaggacgccaacaaagtgcgcgaggacctggcccggtttaagcagcagtctcagcaggtgctggaagtgatgggcctggaacggaacaacgtgaccctgcacatggctaacaccttcgccaccatcaccgtgcccgagagctggctggatatggtgcgagtgggcggcatcttctacggcgacacaatcgccagcaccgactacaagagagtgatgaccttcaagagcaatatcgcctccatcaactactaccccaagggcaacaccgtgggctacgacagaacctacaccctgaagagggacagcgtgctggctaacatccccgtgggatacgccgacggctacagaagagtgttcagcaacgccggccacgccctgatcgctggacagagggtgccagtgctgggaaagaccagcatgaacaccgtgatcgtggacatcaccagcctgaacaacatcaagcccggcgacgaggtggtgttcttcggcaagcagggcaacagcgagatcaccgccgaggaaatcgaggacatctctggcgccctgttcaccgagatgagcatcctgtggggcgccaccaatcagcgcgtgctggtggatcatcatcaccatcaccactga

SEQ ID NO: 14 - protein, 395 aa, Proteus mirabilis
MQQPVNYNPPATQVAQVQPAIVNNSWIEISRSALDFNVKKVQSLLGKQSSLCAVLKGDAYGHDLSLVAPIMIENNVKCIGVTNNQELKEVRDLGFKGRLMRVRNATEQEMAQATNYNVEELIGDLDMAKRLDAIAKQQNKVIPIHLALNSGGMSRNGLEVDNKSGLEKAKQISQLANLKVVGIMSHYPEEDANKVREDLARFKQQSQQVLEVMGLERNNVTLHMANTFATITVPESWLDMVRVGGIFYGDTIASTDYKRVMTFKSNIASINYYPKGNTVGYDRTYTLKRDSVLANIPVGYADGYRRVFSNAGHALIAGQRVPVLGKTSMNTVIVDITSLNNIKPGDEVVFFGKQGNSEITAEEIEDISGALFTEMSILWGATNQRVLVDHHHHHH